(19) United States
(12) Patent Application Publication
(10) Pub. No.: US 2025/0041499 A1
(43) Pub. Date: Feb. 6, 2025

(54) DISPOSABLE SET AND EXTRACORPOREAL BLOOD TREATMENT APPARATUS FOR PREVENTING DEGASSING IN AN INFUSION FLUID LINE

(71) Applicant: GAMBRO LUNDIA AB, Lund (SE)

(72) Inventor: Dominique Pouchoulin, Tramoyes (FR)

(21) Appl. No.: 18/717,278

(22) PCT Filed: Dec. 8, 2022
(86) PCT No.: PCT/EP2022/085079

§ 371 Date: Jun. 6, 2024

(30) Foreign Application Priority Data
Dec. 9, 2021 (EP) 21213423.3

(51) Int. Cl. A61M 1/36; A61M 1/34; A61M 39/10; A61M 39/24; A61M 39/28; A61M 60/113; A61M 60/279; A61M 60/37; A61M 60/546

(52) U.S. Cl. CPC A61M 1/3639; A61M 1/3401; A61M 1/36225; A61M 1/362265; A61M 39/10; A61M 39/28; A61M 60/113; A61M 60/279; A61M 60/37; A61M 60/546; A61M 2039/242; A61M 2039/2433

(57) ABSTRACT

Disposable set for an extracorporeal blood treatment apparatus including a blood circuit comprising a filtration unit, a blood withdrawal line including a blood pump tract configured to be engaged by a blood pump, a blood return line, and an infusion line extending between a first end connected to the blood withdrawal line upstream the blood pump tract, and a second end for connection to an infusion substance source: the blood pump tract is interposed between the filtration unit and the first end of the infusion line. The disposable set further comprises a pressure damper arranged towards or at the first end of the infusion line, the pressure damper being configured to prevent, or reduce an amount of, the access negative pressure to extend in the infusion line upstream the pressure damper.

### FIELD OF THE INVENTION

The present invention relates to a disposable set and to an extracorporeal blood treatment apparatus for preventing degassing in an infusion fluid to be infused in an extracorporeal blood circuit. The present invention particularly relates to extracorporeal blood treatments, such as dialysis, blood oxygenation/CO₂ removal treatments, therapeutic plasma exchange and/or hemoperfusion, wherein a fluid is infused in the extracorporeal blood circuit at a junction point where negative pressure (pressure below external pressure, e.g. atmospheric pressure) occurs.

### BACKGROUND

The kidneys fulfil many functions, including the removal of water, the excretion of catabolites (or waste from the metabolism, for example urea and creatinine), the regulation of the concentration of the electrolytes in the blood (e.g. sodium, potassium, magnesium, calcium, bicarbonates, phosphates, chlorides) and the regulation of the acid/base equilibrium within the body, which is obtained in particular by the removal of weak acids and by the production of ammonium salts.

In individuals who have lost the use of their kidneys, since these excretion and regulation mechanisms no longer work, the body accumulates water and waste from the metabolism and exhibits an excess of metabolites. In order to overcome renal dysfunction, resort is conventionally made to a blood treatment involving extracorporeal circulation within a blood circuit through an exchanger having a semipermeable membrane (dialyzer) in which the patient's blood is circulated on one side of the membrane and a dialysis liquid, comprising the main electrolytes of the blood in concentrations close to those in the blood of a healthy subject, is circulated on the other side. The patient is connected to the extracorporeal blood circuit through a withdrawal (or arterial) and a return (or venous) line, the latter having respective needles at the end portions.

A pressure difference is created between the two compartments of the dialyzer which are delimited by the semipermeable membrane, so that a fraction of the plasma fluid passes by ultrafiltration through the membrane into the compartment containing the dialysis liquid. The blood treatment which takes place in a dialyzer as regards waste from the metabolism and electrolytes results from two mechanisms of molecular transport through the membrane between the blood and the dialysis fluid.

The dialysis fluid is prepared upstream of the dialyzer by mixing pure water with a plurality of predetermined substances, such as electrolytes and buffer, to be exchanged within the dialyzer with the patient's blood. Water comes from an online port receiving purified and de-ionized tap water (e.g., by reverse osmosis), to be subsequently further filtered within the apparatus so that a substantially endless water source is provided to the blood treatment apparatus for subsequent mixing with concentrates. Alternatively, the dialysis fluid is pre-prepared and housed in respective bags to feed the dialysis apparatus, i.e. in case of acute dialysis treatments. Treatment apparatuses configured to deliver hemofiltration or hemodiafiltration treatments or blood oxygenation/CO₂ removal treatments generally comprise an infusion line connected to the blood circuit of the disposable set: the infusion line may be used for infusing replacement fluid into the patient extracorporeal blood and/or to infuse one or more specific substances to control other blood parameters. For example, a bicarbonate solution may be infused into the blood circuit for controlling blood acid-base balance of the patient during the dialysis treatment: a bag containing a bicarbonate liquid solution is usually provided to be connected to the blood circuit in order to allow a controlled infusion. Furthermore, an anticoagulant solution, such as citrate, may be infused in the blood circuit in order to avoid blood coagulation: a bag containing a citrate or other regional anticoagulant is usually provided to be connected to the blood circuit in order to allow a controlled infusion.

Currently, the infusion of certain substances is provided upstream the blood pump on the withdrawal blood line. For example, a citrate solution is usually infused upstream the blood pump to achieve the local anticoagulation effects as soon as possible in the blood circuit. During a dialysis treatment, the blood pump generates a blood flow in the blood circuit; for example, a peristaltic pump conveys fluid by alternately squeezing and releasing the flexible tube of the pump, thereby moving the blood along an advancement direction. A pressure regimen is defined inside the blood circuit, wherein (usually) a negative pressure occurs upstream the blood pump and a positive pressure occurs downstream the blood pump. Of course, the circuit pressures in the blood lines are the consequences of several factors including (but not limited to) the blood pump pumping the fluid, the circuit pressure drops and pressure conditions at the line ends. Blood is withdrawn from a patient access through the withdrawal line and the blood pump generates the blood flow that (usually) contributes to determine a positive pressure regimen, namely a prevalence, downstream the pump, and a negative pressure regimen upstream the pump. The negative pressure, namely a pressure lower than the atmospheric pressure, sometimes occurs at the pre-blood pump (PBP) infusion line that has an injection point into the blood withdrawal line upstream the blood pump. Such negative pressure may cause or promote generation of gases, i.e. bubbles, previously dissolved in the fluid. Solutions for PBP (Pre Blood Pump) infusion, more particularly (but not exclusively) those containing bicarbonate, delivered e.g., during CRRT therapies, degas in case they are infused in negative pressure conditions. Some degassing may also be visually observed in the blood set along the PBP infusion line, from the outlet of the PBP pump to the PBP-access junction connector.

Degassing of solutions along the PBP circuit, from the outlet of the PBP pump to the PBP solution injection site, is at the origin of numerous complaints dealing with both simple presence of air bubbles in the circuit (e.g., suspicion of leak) and/or accumulation of air and/or foam in the deaeration chamber requesting regular monitoring and adjustments.

Notably, the infusion fluids may lead very easily to gas generation, alias degassing, when exposed to the low pressure occurring upstream the blood pump: a degassing chamber arranged downstream the blood pump may be provided to trap the air bubbles, thereby reducing the health risks for the patient: anyhow, the degassing chamber is ineffective to prevent the cause of the fluid degassing process. In addition, the more is the amount of bubbles in the blood circuit, the more is the time frequency requested to periodically service the degassing chamber. Accumulation of foam in the deaeration chamber may lead to various failures when in excessive amounts (including loss of circuit and failure to return blood). Further, when assuming development of some blood activation at blood-air interface, a clotting risk increases.

### OBJECTIVE OF THE INVENTION

The objective of this invention is therefore to at least partially solve one or more of the drawbacks described above.

A first objective is to provide a disposable set and an apparatus for reducing or preventing degassing in an infusion fluid when the latter is infused in a blood line upstream the blood pump in an extracorporeal blood treatment apparatus.

A further aim is to increase safety level for the patient health, reducing risk of embolism and/or blood clotting.

A further objective is to reduce the workload of the medical personnel in servicing the degassing chamber, in order to reduce required interventions.

An aim is to increase the reliability of automatic level management in the chamber and further to decrease field problems and complaints related to degassing of solutions.

A further objective is to provide a device for preventing degassing in an infusion fluid and, at the same time, keeping manufacturing costs of the device low.

At least one of the above objectives is achieved by the present invention.

These objects and more, which will appear more from the following description, are substantially achieved by a disposable set and an apparatus in accordance with one or more of the following claims and/or aspects.

### SUMMARY SECTION

A 1^{st} aspect refers to a disposable set for an extracorporeal blood treatment apparatus (1), the disposable set (100) comprising:
a filtration unit (2);
a blood circuit (17) comprising:
   a blood withdrawal line (6) extending between a first end (6a) connected to the filtration unit (2) and a second end (6b),
   a blood return line (7) extending between a first end (7a) connected to the filtration unit (2) and a second end (7b);
the blood circuit (17), and in particular the blood withdrawal line (6), including a blood pump tract (6p) configured to be engaged by a blood pump (21) of the extracorporeal blood treatment apparatus (1) which is configured to determine a blood flow, at least during an operating condition, an access negative pressure is experienced upstream said blood pump tract (6p), said blood flow in the blood circuit (17) being in a direction from the blood withdrawal line (6) towards the filtration unit (2) and from the filtration unit (2) through the blood return line (7);
an infusion line (51) extending between a first end (51a) connected to the blood circuit (17) at a fluid access, and in particular to the blood withdrawal line (6), upstream the blood pump tract (6p), and a second end (51b) for connection to an infusion substance source (10), wherein the blood pump tract (6p) is interposed between the second end (7b) of the blood return line (7), in particular the filtration unit (2), and the first end of the infusion line (51);
   wherein the disposable set further comprises a pressure damper (40) arranged proximate or at the first end of the infusion line (51), the pressure damper (40) being configured to prevent, or reduce an amount of, the access negative pressure to extend in the infusion line (51) upstream the pressure damper (40).

In an aspect according to the previous aspect, the disposable set further comprises a post infusion line (69) extending between a first end connected to the blood circuit (17) at a fluid access, and in particular to the blood return line (7), downstream the blood pump tract (6p), and a second end (63) for connection to an infusion substance source (64), wherein the blood pump tract (6p) is interposed between the second end (7b) of the blood return line (7), in particular the filtration unit (2), and the first end of the post infusion line (69). In this case, the disposable set may comprise an additional pressure damper arranged proximate or at the first end of the post infusion line (69), the additional pressure damper being configured to prevent, or reduce an amount of, a return negative pressure to extend in the post infusion line (69) upstream the additional pressure damper.

The additional pressure damper downstream the blood pump may be in accordance with any of the subsequent aspects detailing the pressure damper (40).

A further independent aspect refers to a disposable set for an extracorporeal blood treatment apparatus (1), the disposable set (100) comprising:
a filtration unit (2);
a blood circuit (17) comprising:
   a blood withdrawal line (6) extending between a first end (6a) connected to the filtration unit (2) and a second end (6b),
   a blood return line (7) extending between a first end (7a) connected to the filtration unit (2) and a second end (7b);
      the blood circuit (17), and in particular the blood withdrawal line (6), including a blood pump tract (6p) configured to be engaged by a blood pump (21) of the extracorporeal blood treatment apparatus (1) which is configured to determine a blood flow, at least during an operating condition, a return negative pressure is experienced downstream the filtration unit (2), said blood flow in the blood circuit (17) being in a direction from the blood withdrawal line (6) towards the filtration unit (2) and from the filtration unit (2) through the blood return line (7);
a post infusion line (69) extending between a first end connected to the blood circuit (17) at a fluid access, and in particular to the blood return line (7), downstream the blood pump tract (6p), and a second end (63) for connection to an infusion substance source (64), wherein the blood pump tract (6p) is interposed between the second end (7b) of the blood return line (7), in particular the filtration unit (2), and the first end of the post infusion line (69);
   wherein the disposable set further comprises a pressure damper (40) arranged proximate or at the first end of the post infusion line (69), the pressure damper (40) being configured to prevent, or reduce an amount of, the return negative pressure to extend in the post infusion line (69) upstream the pressure damper (40).

In an aspect according to any of the previous aspects, the second end (6b) of the blood withdrawal line (6) and the second end (7b) of the blood return line (7) are:
for connection to a patient; or
for connection/connected to a blood duct (306, 307) of a further extracorporeal blood circuit (such as an extra corporeal membrane oxygenation (ECMO) circuit (300)).

In a 2^{nd} aspect according to any one of the preceding aspects, the pressure damper (40) comprises a flow passage restrictor (41) wherein:
the flow passage restrictor (41) has a damper passage section configured to allow the infusion fluid to pass through, and
the infusion line (51) has a respective fluid passage section, in particular an average fluid passage section, configured to allow the infusion fluid to pass through,
wherein the damper passage section is smaller than the fluid passage section, optionally wherein the damper passage section is at least 50% smaller than the fluid passage section, in particular at least the 80% smaller, more in particular at least the 90% smaller.

In a 3^{rd} aspect according to any one of the preceding aspects, the pressure damper (40) comprises a one-way valve (46) configured to allow fluid passage only in an infusion direction directed from the infusion line (51) towards the blood circuit (17), in particular towards the withdrawal line (17), and configured to move between an open position, in which the fluid passage is allowed in said infusion direction, and a closed position, in which fluid passage is prevented in both directions, in particular wherein fluid passage in a direction from the withdrawal line (6) to the infusion line (51) is prevented in any conditions.

In a 4^{th} aspect according to the 3° aspect, the one-way valve (46) is preset at an opening pressure threshold to switch between the closed and the open position and vice versa.

In a 5^{th} aspect according to the 3° aspect, the one-way valve (46) is preset at an opening pressure threshold to switch between the closed and the open position and vice versa, so that:
if a differential pressure between an upstream section and a downstream section of the one-way valve (46) is equal or higher than said opening pressure threshold, the one-way valve (46) is configured to switch to or to maintain the open position,
in particular wherein the differential pressure is defined between an high pressure zone upstream the one-way valve (46), and a low pressure zone downstream one-way valve (46) according to the infusion direction, in particular said high pressure being higher than said low pressure,
and
if said differential pressure is lower than said opening pressure threshold, the one-way valve (46) is configured to switch to or to maintain the closed position.

In a 6^{th} aspect according to any one of preceding aspects from 3 to 5, the pressure damper (40) comprises said one-way valve (46), and wherein the preset opening pressure threshold of the one-way valve (46) corresponds to a differential pressure value higher than zero mmHg, in particular higher than 160 mmHg and particularly comprised between 160 and 500 mmHg, in particular between 190 and 450 mmHg, more in particular between 200 and 400 mmHg, more in detail between 240 and 350 mmHg.

In a 6^{th} bis aspect according to the two preceding aspects, the opening pressure threshold of the one-way valve (46) is set about a maximum negative pressure allowed at the fluid access during standard working condition of the extracorporeal blood treatment apparatus (1). “About” is intended within +/−100 mmHg, particularly within +/−50 mmHg.

In a 6^{th} ter aspect according to the preceding aspect, the maximum allowed negative pressure is defined as a difference between the atmospheric pressure and minimal pressure reached in the blood line at the fluid access of the infusion line (51) during standard working condition of the extracorporeal blood treatment apparatus (1).

In a 7^{th} aspect according to any one of preceding aspects from 3, the pressure damper (40) comprises said one-way valve (46), and wherein the one-way valve (46) comprises an internal diaphragm (46a) movable between the open position and the closed position, the internal diaphragm (46a) being preloaded in the closed position, said preload defining the preset opening pressure threshold, in particular the internal diaphragm being a flexible membrane made of a material between PVC, silicone, rubber or the like.

In a 8^{th} aspect according to any one of preceding aspects from 3, said one-way valve (46) is arranged on the infusion line (51) at a distance from the first end of the infusion line (51) no longer than 6 cm, optionally no longer that 3 cm, in particular comprised between 0.1 cm and 6 cm, more in particular between 0.2 and 3 cm.

In a 9° aspect according to any one of preceding aspects from 3, the disposable set comprises a Luer lock connector (47) housing said one-way valve (46), in particular wherein said internal diaphragm (46a) is within an internal fluid passage of the Luer lock connector.

In a 9^{th} bis aspect according to any one of preceding aspects from 3, when the differential pressure is below said preset opening pressure threshold, the one-way valve is in the closed position.

In a 10^{th} aspect according to the aspect 2, in a condition wherein a fluid flows through the pressure damper (40), the pressure damper (40) is configured to determine a local pressure drop along a flow passage extension of the pressure damper,

in particular wherein said local pressure drop is (preferably much) higher than a pressure drop defined by the infusion line (51) along the same length extension of the flow passage extension of the pressure damper (40).

In a 10^{th} bis aspect according to the aspect 2, the pressure damper (40) is the flow passage restrictor (41), and wherein:
the damper passage extends by a length comprised between 30 mm and 200 mm, in particular between 40 mm and 160 mm, more in particular between 55 mm and 150 mm, and
the damper passage section extends by a diameter comprised between 0.3 mm and 0.6 mm, in particular between 0.35 mm and 0.55 mm.

In an 11^{th} aspect according to any one of preceding aspects 2 and 10:
if the damper passage section has a diameter substantially equal to 0.5 mm, the damper passage extends by a length substantially equal to 145 mm optionally ±15 mm, and/or
if the damper passage section has a diameter substantially equal to 0.4 mm, the damper passage extends by a length substantially equal to 60 mm optionally ±10 mm,
   in particular wherein said diameter is substantially constant over said length.

In a 12^{th} aspect according to any one of preceding aspects 2 and 10-11, the flow passage restrictor (41) comprises a flexible tube tract, in particular said tube tract being made of PVC, silicone, or biocompatible flexible material.

In a 13^{th} aspect according to any one of preceding aspects 2 and 10-12, the flow passage restrictor (41) comprises a septum defining the damper passage section,

optionally the damper passage section axially extending:
along the fluid flow direction of the infusion line (51) by a damper passage length comprised between 1 mm and 30 mm, in particular between 2 mm and 20 mm, in particular between 4 mm and 10 mm, and
radially by a diameter comprised between 0.2 mm and 3 mm;
optionally wherein the damper passage section is substantially constant along said damper passage length.

In a 14^{th} aspect according to any one of preceding aspects, the disposable set comprises an infusion connector (48) including:
a connector body defining an internal volume;
a blood inlet and a blood outlet fluidly communicating each other and with said internal volume, the blood inlet being connected to the blood withdrawal line (6) facing the second end of the blood withdrawal line, and the blood outlet being connected to the blood withdrawal line (6) facing the blood pump tract (6p) of the blood withdrawal line (6);
an infusion inlet fluidly communicating with said internal volume and connected to the first end of the infusion line (51).

In a 15^{th} aspect according to the preceding aspect, the infusion connector is a three-way connector, in particular having two inlets and one outlet.

In a 16^{th} aspect according to any one of preceding two aspects, the pressure damper (40) is arranged within the internal volume of the connector body, optionally in the infusion inlet of the infusion connector, in particular the pressure damper (40) comprising the flow passage restrictor (41) or the one-way valve (46).

In a 16^{th} bis aspect according to any one of preceding three aspects, the infusion connector is welded or glued to the infusion line (51) and to the blood line.

In a 16^{th} ter aspect according to any one of preceding four aspects, the infusion connector is irremovably coupled to the infusion line (51) and to the blood line.

In a 17^{th} aspect according to any one of preceding aspects, the infusion line (51) comprises a respective infusion pump tract (51p) interposed between the first end and the second end of the infusion line (51), the infusion pump tract of the infusion line (51) being configured to be engaged by an infusion peristaltic pump configured to deliver a blood flow, at least during an operating condition, a positive pressure regimen is present downstream the infusion pump tract to allow the infusion fluid to flow in a direction towards the first end of the infusion line (51) and towards the blood withdrawal line (6).

In an 18^{th} aspect according to any one of preceding aspect, the pressure damper (40) is arranged on the infusion line (51) between the infusion pump tract (51p) and the blood circuit (17), in particular between the infusion pump tract (51p) and the blood withdrawal line (6).

In a 19^{th} aspect according to any one of preceding aspects, the infusion inlet of the infusion connector is part of the infusion line (51).

In a 20^{th} aspect according to any one of preceding aspects, the infusion connector is stiffer than the infusion line (51) and/or of the blood withdrawal line (6).

In a 21^{st} aspect according to any one of preceding aspects, the infusion line (51) is welded or glued to the infusion connector, in particular at a coupling portion.

In a 22^{nd} aspect according to any one of preceding aspects, the infusion line (51) is connected to the infusion connector at a coupling portion, wherein an internal flow passage is substantially constant from the infusion line (51) up to the infusion connector, including the coupling portion, in particular wherein the coupling portion does not define a discontinuity in the internal flow passage.

In a 23^{rd} aspect according to any one of preceding aspects, the infusion line (51) defines an internal flow passage having a fluid passage section substantially constant up to the pressure damper (40), the latter defining a discontinuity in the fluid passage section.

In a 24^{th} aspect according to any one of preceding aspects, the infusion line (51) is flexible, in particular more flexible than the infusion connector.

In a 25^{th} aspect according to any one of preceding aspects, the blood withdrawal line (6) is flexible, in particular more flexible than the infusion connector.

In a 26^{th} aspect according to any one of preceding aspects, the blood return line (7) is flexible, in particular more flexible than the infusion connector.

In a 27^{th} aspect according to any one of preceding aspects, the blood withdrawal line (6) of the blood circuit has a fluid passage section comprised between 3 mm² and 20 mm² or a diameter comprised between 2 mm and 5 mm.

In a 28^{th} aspect according to any one of preceding aspects, the infusion line (51) has a fluid passage section comprised between a diameter comprised between 1 mm and 4 (or 3) mm.

In a 29^{th} aspect according to any one of preceding aspects, the flow passage restrictor (41) of the pressure damper (40) has a fluid passage section comprised between 0.07 mm² and 0.28 mm² or a diameter comprised between 0.3 mm and 0.6 mm, more particularly a diameter comprised between 0.4 mm and 0.5 mm.

In a 30^{th} aspect according to any one of preceding aspects, the flow passage restrictor (41) of the pressure damper (40) extends by a length comprised between 20 mm and 200 mm or a length comprised between 55 mm and 150 mm.

In a 31^{st} aspect according to any one of preceding aspects, the flow passage restrictor (41) of the pressure damper (40) defines a sharp discontinuity for the fluid flow in the infusion line (51).

In a 32^{nd} aspect according to any one of preceding aspects, the flow passage restrictor (41) or the one-way valve (46) are configured to define a pressure drop in the infusion fluid during an operating condition of the treatment apparatus, in particular when an infusion fluid flows within the infusion line (51) towards the blood withdrawal line (6).

In a 33^{rd} aspect according to any one of preceding aspects, the disposable set further includes a fluid circuit comprising:
an effluent fluid line (13) extending between a first end connected to an outlet of a secondary chamber (4) of the filtration unit (2), and a second end for connection to a drain or a collection container (62),
optionally a dialysis liquid supply line (8) extended between a first end connected to an inlet of a secondary chamber of the filtration unit (2), and a second end for connection to a dialysis fluid source, for example to a bag or to a dialysis preparation unit.

In a 34^{th} aspect according to any one of preceding aspects, the dialysis liquid supply line (8) comprises a respective supply pump tract configured to be engaged to a dialysis fluid pump (25), and wherein the effluent fluid line (13) comprises a respective pump tract configured to be engaged to a dialysate pump (26).

In a 35^{th} aspect according to any one of preceding aspects, the disposable set comprises the infusion substance source (10) connected to the second end of the infusion line (51), said infusion substance source (10) comprising an infusion bag housing a fluid infusion solution, in particular said infusion solution comprising one of a replacement fluid, saline, and a regional anticoagulant solution, optionally said infusion solution comprising one or more components between bicarbonate, acetate, lactate, citrate, and electrolytes.

In a 36^{th} aspect according to any one of preceding aspects, the one-way valve (46) is arranged on the infusion line (51) upstream the infusion connector, in particular the one-way valve (46) being arranged close to the infusion connector in particular at a distance from the infusion connector comprised between 0.1 cm and 6 cm, more in particular between 0.2 and 3 cm.

In a 37^{th} aspect according to any one of preceding aspects, the filtration unit (2) has a primary chamber (3) and a secondary chamber (4) separated by a semi-permeable membrane (5).

In a 38^{th} aspect according to any one of preceding aspects, wherein the access negative pressure is a pressure lower than the atmospheric pressure, the positive pressure is a pressure higher than the atmospheric pressure.

In a 39^{th} aspect according to any one of preceding aspects, the flow passage restrictor (41) defines an aperture, which put the infusion line (51) in fluid communication with the blood withdrawal line (6), said aperture being fixed in size in particular said aperture being not variable and not varying said size during an operating condition of the extracorporeal blood treatment apparatus.

In a 40^{th} aspect according to any one of preceding aspects, the flow passage restrictor (41) is defined by a restricting portion of the infusion line (51), said restricting portion of the infusion fluid being configured to be pinched by a clamp.

In a 40^{th} bis aspect according to any one of preceding aspects, the access negative pressure is the actual blood pressure at the junction point between the blood line (6, 7) and the infusion line (51).

A 41^{st} aspect is directed to an assembly comprising:
a disposable set for an extracorporeal blood treatment apparatus, the disposable set comprising:
   a filtration unit (2);
   a blood circuit (17) comprising a blood withdrawal line (6) extending between a first end connected to the filtration unit (2) and a second end, e.g., for connection to a patient (P) or to a further blood circuit, and a blood return line (7) extending between a first end connected to the filtration unit (2) and a second end, e.g., for connection to said patient (P) or to said further blood circuit;
   the blood circuit (17), and in particular the blood withdrawal line (6), including a pump tract configured to be engaged to a blood pump (21) of the extracorporeal blood treatment apparatus which is configured to determine a blood flow, at least during an operating condition, a positive pressure is present downstream the blood pump tract (6p) and an access negative pressure is present upstream said pump tract, said blood flow in the blood circuit being in a direction from the blood withdrawal line (6) towards the filtration unit (2) and from the filtration unit (2) through the blood return line (7);
   an infusion line (51) extending between a first end connected to the blood circuit (17) at a fluid access, in particular to the blood withdrawal line (6), upstream the blood pump tract (6p), and a second end for connection to an infusion substance source (10), wherein the blood pump tract (6p) is interposed between the second end (7b) of the blood return line (7), and in particular the filtration unit (2), and the first end of the infusion line (51);
      and
a clamp (80) configured to engage a portion of the infusion line (51) and to reduce, without closing, an internal lumen thereof, said clamp comprising at least one between:
   a portable clamp (81) comprising a clamping seat configured to receive a portion of the infusion line (51) and having a fixed size to determine a predefined lumen reduction of the infusion line (51);
   a variable clamp comprising a clamping seat configured to receive a portion of the infusion line (51) and having a regulator for varying the lumen reduction of the infusion line (51),
      in a condition wherein the clamp is not active on the infusion line, the infusion line (51) has a respective fluid passage section, in particular an average fluid passage section, configured to allow the infusion fluid to pass through, in a condition wherein the clamp is engaged on the infusion line and active to reduce the internal lumen thereof, the infusion line (51) has a damper passage section configured to allow the infusion fluid to pass through, and wherein the damper passage section is smaller than the fluid passage section, optionally wherein the damper passage section is not less than 50% smaller than the fluid passage section, in particular not less than 80% smaller, more in particular not less than 90% smaller.

A 41^{st} bis aspect is directed to an assembly comprising:
a disposable set disposable set for an extracorporeal blood treatment apparatus (1), the disposable set (100) comprising:
   a filtration unit (2);
   a blood circuit (17) comprising:
      a blood withdrawal line (6) extending between a first end (6a) connected to the filtration unit (2) and a second end (6b),
      a blood return line (7) extending between a first end (7a) connected to the filtration unit (2) and a second end (7b);
   the blood circuit (17), and in particular the blood withdrawal line (6), including a blood pump tract (6p) configured to be engaged by a blood pump (21) of the extracorporeal blood treatment apparatus (1) which is configured to determine a blood flow, at least during an operating condition, a return negative pressure is experienced downstream the filtration unit (2), said blood flow in the blood circuit (17) being in a direction from the blood withdrawal line (6) towards the filtration unit (2) and from the filtration unit (2) through the blood return line (7);
   a post infusion line (69) extending between a first end connected to the blood circuit (17) at a fluid access, and in particular to the blood return line (7), downstream the blood pump tract (6p), and a second end (63) for connection to an infusion substance source (64), wherein the blood pump tract (6p) is interposed between the second end (7b) of the blood return line (7), in particular the filtration unit (2), and the first end of the post infusion line (69);
      and
a clamp (80) configured to engage a portion of the post infusion line (69) and to reduce, without closing, an internal lumen thereof, said clamp comprising at least one between:
   a portable clamp (81) comprising a clamping seat configured to receive a portion of the post infusion line (69) and having a fixed size to determine a predefined lumen reduction of the post infusion line (69);
   a variable clamp comprising a clamping seat configured to receive a portion of the post infusion line (69) and having a regulator for varying the lumen reduction of the post infusion line (69),
      in a condition wherein the clamp is not active on the post infusion line, the post infusion line (69) has a respective fluid passage section, in particular an average fluid passage section, configured to allow the post infusion fluid to pass through,
      in a condition wherein the clamp is engaged on the post infusion line and active to reduce the internal lumen thereof, the post infusion line (69) has a damper passage section configured to allow the post infusion fluid to pass through, and
      wherein the damper passage section is smaller than the fluid passage section, optionally wherein the damper passage section is not less than 50% smaller than the fluid passage section, in particular not less than 80% smaller, more in particular not less than 90% smaller.

A 42^{nd} aspect is directed to an assembly comprising:
a disposable set for an extracorporeal blood treatment apparatus according to any one of the preceding aspects, and
a clamp (80) configured to engage a portion of the infusion line (51) and/or post infusion line (69) and to reduce, without closing, an internal lumen thereof.

In a 43^{rd} aspect according to the preceding aspect, said clamp comprises at least one between:
a portable clamp (81) comprising a clamping seat configured to receive a portion of the infusion line (51) and having a fixed size to determine a predefined lumen reduction of the infusion line (51);
a variable clamp comprising a clamping seat configured to receive a portion of the infusion line (51) and having a regulator for varying the lumen reduction of the infusion line (51),
   in a condition wherein the clamp is not active on the infusion line, the infusion line (51) has a respective fluid passage section, in particular an average fluid passage section, configured to allow the infusion fluid to pass through,
   in a condition wherein the clamp is engaged on the infusion line and active to reduce the internal lumen thereof, the infusion line (51) has a damper passage section configured to allow the infusion fluid to pass through, and in particular wherein the damper passage section is smaller than the fluid passage section, optionally wherein the damper passage section is not less than 50% smaller than the fluid passage section, in particular not less than 80% smaller, more in particular not less than 90% smaller.

A 44^{th} aspect is directed to an extracorporeal blood treatment apparatus comprising:
the disposable set according to any one of the preceding aspects, and
the blood pump (21),
a control unit operatively connected to the blood pump (21) and configured to control a blood flow rate of the blood pump (21).

A 45^{th} aspect is directed to an extracorporeal blood treatment apparatus comprising:
the disposable set according to any one of the preceding aspects 1-40, or the assembly of the preceding aspects 41-43,
a blood pump cooperating with the blood pump tract (6p) of the blood withdrawal line (6),
an infusion pump (54) cooperating with an infusion pump tract (51p) of the infusion line (51), said infusion pump (54) being arranged between the second end of the infusion line (51) and the pressure damper (40), in particular upstream the pressure damper (40) and downstream the infusion substance source (10),
a control unit (12) operatively connected to the blood pump (21) and the infusion pump (54), the control unit (12) being configured to control the blood pump (21) and the infusion pump (54) at a corresponding blood flow rate and an infusion flow rate.

In a 46^{th} aspect according to the preceding aspect, the control unit is configured to define a treatment condition wherein:
the blood flow rate is comprised between 50 ml/min and 600 ml/min, in particular between 50 ml/min and 350 ml/min, more in particular between 100 ml/min and 300 ml/min, and/or
the infusion flow rate is comprised between 200 ml/h and 4000 ml/h, in particular higher than 500 ml/h and lower than 2000 ml/h.

In a 47^{th} aspect according to any one of preceding aspects, the pressure damper (40) comprises the one-way valve (46), and wherein the control unit is configured to define:
a first condition wherein the infusion pump (54) is active to determine a fluid flow, an infusion positive pressure being present downstream the infusion pump, and the blood pump is active to determine a blood flow, an access negative pressure being present upstream the blood pump and a positive pressure being present downstream the blood pump,
wherein a pressure difference between said infusion positive pressure and the access negative pressure is higher than the preset opening pressure threshold of the one-way valve (46) to allow the infusion fluid to enter the blood withdrawal line (6); and
a second condition wherein the infusion pump (54) is stopped to prevent infusion of the infusion fluid into the blood withdrawal line (6), and the blood pump (21) is active to determine blood flow in the blood circuit, in said second condition the one-way valve (46) is closed as a difference between a pressure in the infusion line (51) and the access negative pressure is lower than the preset opening pressure threshold of the one-way valve (46), in particular preventing the access negative pressure to extend in the infusion line (51).

With regard to the one-way valve, the system is not dependent on the infusion line (specifically the Pre-Blood-Pump infusion line) flow rate. Valve opening pressure is built in the design and the pressure drop through the valve is neglected.

The opening pressure is selected to be higher than the (absolute value of the) minimal access pressure of the defined/designed access pressure operating range.

For example, minimum access pressure may be set to −250 mmHg; consequently the one-way valve (46) has ideally an opening pressure (slightly) above +250 mmHg as to obtain:

$${P}_{acc \_ min} + {P}_{opening} > 0$$

Namely, the minimum access pressure plus the one-way valve (46) opening pressure is higher than zero.

When the infusion stops the one-way valve (46) closes as soon as the infusion circuit pressure becomes lower than the sum of the access pressure and opening pressure (P_{access}+P_{opening}). Given to the circuit compliance, this happens with a (very) short delay after the infusion pump stop. Then, if the access pressure decreases, a very small amount of fluid will flow through the valve in order to keep infusion circuit pressure equaled to P_{access}+P_{opening}. However, at the same time no fluid/blood may flow ‘up’ from the blood line to the infusion line. In short, when the infusion pump is stopped, and if the pump is occlusive, the pressure upstream the valve will ‘record’ the lowest access pressure that will occur during the infusion pump stop. Once the infusion pump is resumed, the infusion will increase back to the current access pressure plus P_{opening}.

Of course, a valve with (slightly) lower opening pressure, e.g., +200 mmHg may also be accepted as few treatments operate below −200 mmHg and fluid degassing will be very low at (low) negative pressures >−50 mmHg.

In more general terms, the opening pressure threshold of the one-way valve 46 may be set about (e.g., within +/−100 mmHg, particularly within +/−50 mmHg) a maximum negative pressure allowed at the fluid access during standard working condition of the extracorporeal blood treatment apparatus (1).

In a 48^{th} aspect according to any one of the preceding aspects from 44 to 47, the control unit is configured to define a treatment condition wherein the blood pump (21) is set at a flow rate comprised between 50 ml/min and 600 ml/min, in particular between 100 ml/min and 350 ml/min, more in particular between 200 ml/min and 300 ml/min.

In a 49^{th} aspect according to an one of the preceding aspects from 44, during said treatment condition, the access negative pressure is present in the blood withdrawal line (6) upstream the blood pump tract (6p), in particular upstream the blood pump.

In a 50^{th} aspect according to any one of the preceding aspects from 44, the infusion pump is operatively connected to the control unit which is configured to selectively:
control the infusion pump to deliver an infusion fluid into the blood withdrawal line (6) in particular at a flow rate comprised between 200 ml/h and 4000 ml/h, in particular between 500 ml/h and 2000 ml/h, or
stop the infusion pump to arrest delivery of the infusion fluid.

In a 51^{st} aspect according to any one of the preceding aspects from 44, the infusion pump is a positive displacement pump, such as a peristaltic pump or a finger pump, or a syringe pump (plunger acting on a syringe).

In a 52^{nd} aspect according to an one of the preceding aspects from 44, the infusion substance source (10) is an infusion bag housing the infusion fluid, the extracorporeal blood treatment apparatus further comprising a scale operatively connected to the control unit and configured to weight the infusion substance source (10), wherein the control unit is configured to emit a signal representative of a weight of the infusion bag and to control the infusion flow rate by commanding the infusion pump (54) as a function of the weight signal.

In a 52^{nd} aspect according to any one of the preceding aspects, the infusion pump (54) is a peristaltic pump.

In a 53^{rd} aspect according to any one of the preceding aspects, the blood pump (21) is a peristaltic pump.

In a 54^{th} aspect according to any one of the preceding aspects, the blood pump tract (6p) is interposed between the filtration unit (2) and the first end of the infusion line (51), in particular between a blood inlet of the filtration unit (2) and the first end of the infusion line (51).

In a 55^{th} aspect according to any one of the preceding aspects, the control unit is configured to control the apparatus to perform one between an ultrafiltration treatment (UF), particularly with regional anticoagulation, a haemodialysis treatment (HD), particularly with regional anticoagulation, a haemofiltration treatment (HF), haemodiafiltration treatment (HDF).

In a 56^{th} aspect according to any one of the preceding aspects 1-54, the control unit is configured to control the apparatus configured for CO₂ removal to perform a CO₂ removal treatment.

In a 57^{th} aspect according to the preceding aspect, the apparatus comprises a CO₂ removal device arranged on the blood return line (7).

In a 58^{th} aspect according to the preceding aspect, the CO₂ removal device is arranged between the blood outlet at the filtration unit (2) and the bubble trap.

In a 59^{th} aspect according to any one of the preceding aspects, an ECMO circuit comprises at least:
a blood withdrawal duct (306) for connection with the vascular access of the patient and configured to receive blood from the patient,
a blood return duct (307) for connection with the vascular access of the patient and configured to return blood to the patient,
a blood pump (321) configured to promote blood flow in the ECMO circuit (300)
an oxygenator (310) arranged on the blood return duct (307), and configured to oxygenate the blood.

In a 60^{th} aspect according to the preceding aspect from 59:
if the infusion line (51) is arranged on the blood withdrawal line (6) and if the blood withdrawal line (6) is connected to the ECMO circuit (300) upstream the blood pump (321) of the ECMO circuit, the pressure damper (40) is located at the fluid access between the blood withdrawal line (6) and the infusion line (51);
if the infusion line (51) is arranged on the blood return line (7) and if the blood return line (6) is connected to the ECMO circuit (300) upstream the blood pump (321) of the ECMO circuit, the pressure damper (40) is located at the fluid access between the blood return line (7) and the infusion line (51).

In a 61^{st} aspect according to any one of the preceding aspects from 59, the pressure damper is arranged on the return line (7) of the disposable set (400) connected to the ECMO circuit (300) where a negative pressure is experienced.

In a 62^{nd} aspect according to any one of the preceding aspects from 59, the blood withdrawal line (6) of the disposable set (100) is connected to the ECMO circuit (300) upstream (or optionally downstream) the blood pump (321) of the ECMO circuit (300).

In a 63^{rd} aspect according to any one of the preceding aspects from 59, the blood return line (7) of the disposable set (100) is connected to the ECMO circuit (300) downstream (or optionally upstream) the blood pump (321) of the ECMO circuit (300).

In a 64^{th} aspect according to any one of the preceding aspects from 59, the blood pump (321) of the ECMO circuit is configured to promote blood flow in the ECMO circuit in a direction from the blood withdrawal duct (306) towards the blood return duct (307), in particular through the oxygenator (310).

In a 65^{th} aspect according to any one of the preceding aspects from 59, the disposable set (100) is connected to the ECMO circuit (300) by a removable connection system.

In a 66^{th} aspect according to any one of the preceding aspects 1-54, the control unit is configured to control the apparatus to perform a therapeutic plasma exchange treatment.

In a 67^{th} aspect according to any one of the preceding aspects, the pressure damper (40) comprises a one-way valve configured to allow fluid passage only in an infusion direction directed from the infusion line (51) towards the blood circuit (17), in particular towards the withdrawal line (17), said one-way valve (46) being configured to move between an open position, in which fluid passage is allowed in said infusion direction, and a closed position, in which fluid passage is prevented in said infusion direction and in an opposite direction, in particular wherein fluid passage in a direction from the blood circuit (17) to the infusion line (51) is prevented, and wherein said one-way valve (46) is a duckbill valve (75).

In a 68^{th} aspect according to the preceding aspect, said duckbill valve (75) comprises a housing (76) defining an inner volume (76a).

In a 69^{th} aspect according to the preceding aspect, the housing (76) comprises:
an inlet connector (75a) connected or configured to be connected to an upstream tract of the infusion line (51);
an outlet connector (75b) connected or configured to be connected to a downstream tract of the infusion line (51) or to the blood circuit (17);
a duckbill component (77) arranged in the inner volume (76a) of the housing (76) and configured to allow the infusion fluid to flow from said inlet connector (75a) to said outlet connector (75b) and to prevent fluid to flow backwards from said outlet connector (75b) to said inlet connector (75a).

In a 70^{th} aspect according to the preceding aspect, the duckbill component (77) is made of a flexible and/or elastic material, optionally said material being one of silicone, silicone based material, rubber, PVC and latex (or equivalent material).

In a 70^{th} bis aspect according to any one of the preceding aspects from 69, said duckbill valve (75) is arranged on the infusion line (51) at a distance from the first end of the infusion line (51) no longer than 6 cm, optionally no longer that 3 cm, in particular comprised between 0 cm and 6 cm, more in particular between 0.2 and 3 cm.

In a 71^{st} aspect according to any one of the preceding aspects from 69, said inlet connector (75a) of the duckbill valve (75) faces the infusion substance source (10).

In a 72^{nd} aspect according to any one of the preceding aspects from 69, said outlet connector (75b) of the duckbill valve (75) faces the blood circuit (17), in particular faces or defines the first end (51a) of the infusion line (51).

In a 73^{rd} aspect according to any one of the preceding aspects from 69, the outlet connector (75b) of the duckbill valve (75) is interposed between the blood circuit (17) and the inlet connector (75a) of the duckbill valve (75) in particular along a fluid path of the infusion fluid.

In a 74^{th} aspect according to any one of the preceding aspects from 69, the duckbill component (77) is interposed between the inlet connector (75a) and the outlet connector (75b) along a fluid path of the infusion fluid.

In a 75^{th} aspect according to any one of the preceding aspects from 69 to 74, the inlet connector (75a) of the duckbill valve (75) is opposite to the outlet connector (75b) of the duckbill valve (75) with respect to the duckbill component (77).

In a 76^{th} aspect according to any one of the preceding aspects from 69 to 75, the inlet connector (75a) and the outlet connector (75b) are substantially aligned along a line in particular a straight line coincident with the infusion fluid passage direction.

In a 77^{th} aspect according to any one of the preceding aspects from 69 to 76, the duckbill component (77) of the duckbill valve (75) has a tapered shape extending between a passage wide section (77a) and a passage closing section (77b) for the fluid.

In a 77^{th} bis aspect according to any one of the preceding aspects from 69 to 77, the duckbill component (77) has lateral walls developing inside the inner volume (76a) of the chamber (76), wherein an external surface of the lateral walls is subject to the pressure present inside the chamber (76) and an internal surface of the lateral walls is subject to the pressure present at the inlet connector (75a).

In a 78^{th} aspect according to the preceding aspect 77, the duckbill valve (75) is configured to allow the infusion fluid to flow sequentially from the inlet connector (75a), into the passage wide section (77a), and then through the passage closing section (77b) of the duckbill component (77), and afterwards through the outlet connector (75b) of the duckbill valve (75).

In a 79^{th} aspect according to any one of the preceding aspects from 77 to 78, the passage wide section (77a) is connected, in particular in a fluid tight manner, to an exit of the inlet connector (75a) inside the inner volume (76a).

In an 80^{th} aspect according to any one of the preceding aspects from 77 to 79, the passage wide section (77a) is interposed between the passage closing section (77b) and the inlet connector (75a) of the duckbill valve (75).

In an 81^{st} aspect according to any one of the preceding aspects from 77, the passage closing section (77b) is arranged downstream of the passage wide section with respect to an/the infusion fluid direction (ID).

In an 82^{nd} aspect according to any one of the preceding aspects from 77, the passage closing section (77b) opens into the inner volume (76a) of the housing (76), in particular wherein the passage closing section (77b) is not directly connected to the outlet connector (75b).

In an 83^{rd} aspect according to any one of the preceding aspects from 77, the passage closing section (77b) is cantilevered within the inner volume (76a) of the housing (76) of the duckbill valve (75).

In an 84^{th} aspect according to any one of the preceding aspects from 77, the passage closing section (77b) comprises a deformable slit deformable between:
an open condition, wherein the passage closing section (77b) of the duckbill component (77) allows the infusion fluid to flow from the inlet connector (75a) towards the outlet connector (75b), in particular said open condition of the slit defining the open position of the one-way valve; and
a closed condition, wherein the passage closing section (77b) of the duckbill component (77) is closed, in particular preventing fluid to flow through the duckbill component (77), in particular said closed condition of the slit defining the closed position of the one-way valve.

In an 85^{th} aspect according to any one of the preceding aspects from 77, the passage closing section (77b) is configured to move from the closed condition to the open condition when a pressure upstream the duckbill component (77) is greater than a pressure downstream the duckbill component (77), in particular at least 3% or at least 5% greater than the pressure downstream the duckbill component (77).

In an 86^{th} aspect according to any one of the preceding aspects from 77, the passage closing section (77b) is made of a flexible and/or elastic material, optionally said material being one of silicone, silicone based material, rubber, PVC, and latex.

In an 87^{th} aspect according to any one of the preceding aspects from 68, the housing (76) is made of a rigid material, optionally a plastic material.

In an 88^{th} aspect according to any one of the preceding aspects from 68, the housing (76) is, at least partially, transparent.

In an 89^{th} aspect according to any one of the preceding aspects, the pressure damper (40) comprises a deformable flow passage restrictor (42) comprising, in particular being made of, an elastically deformable material, optionally one of silicone, silicone based material, rubber, PVC, and latex, and wherein said deformable flow passage restrictor (42) is configurable:
in a rest condition, wherein the deformable flow passage restrictor (42) has a damper lumen (44) which is substantially closed to prevent fluid flow or which defines a damper passage section for the infusion fluid having a size lower than 1 mm², in particular lower than 0,5 mm², more in particular lower than 0,1 mm², more in particular lower than 0,01 mm²,
in particular in the rest condition the damper lumen (44) of the deformable flow passage restrictor (42) is substantially closed in a fluid tight manner;
in an infusion condition, when a lumen pressure inside the deformable flow passage restrictor (42) is above a predefined threshold, the damper lumen (44) of the deformable flow passage restrictor (42) opens to allow transit of the infusion fluid.

Notably, the deformable flow passage restrictor (42) has to prevent pressures below atmospheric pressure that may be generated along the blood circuit (17) to reach the infusion line sections upstream the dumper. In the configuration above claimed any pressure below atmospheric pressure tends to close the damper lumen (44) thereby preventing transfer of such a pressure.

In a 90^{th} aspect according to the preceding aspect, said predefined threshold to open the damper lumen (44) is greater than 1,02 times the atmospheric pressure, in particular greater than 1,05 times the atmospheric pressure, in particular greater than 1,1 or 1,2 times the atmospheric pressure.

In a 91^{st} aspect according to any one of the preceding aspects from 89 to 90, said predefined threshold to open the damper lumen (44) is greater than 0.05 Bar or 0.1 Bar, optionally greater than 0.2 Bar or 0,3 Bar, said predefined threshold being a relative pressure to the atmospheric pressure.

In a 92^{nd} aspect according to any one of the preceding aspects from 89, the damper lumen (44) of the deformable flow passage restrictor (42) extends in a flow direction by a length comprised between 5 mm and 100 mm, in particular between 10 mm and 50 mm, optionally between 10 mm and 30 mm.

In a 93^{rd} aspect according to any one of the preceding aspects from 89, the deformable flow passage restrictor (42) has, in the rest condition, a collapsed damper lumen (44) having a shape in section between a line, optionally a straight or a curved line, and a dot.

In a 93^{rd} bis aspect according to any one of the preceding aspects from 89 to 92, the damper lumen (44) of the deformable flow passage restrictor (42) is normally closed, in particular wherein the damper lumen (44) of the deformable flow passage restrictor (42) is normally in the rest condition.

In a 94^{th} aspect according to any one of the preceding aspects from 89 to 93, if the deformable flow passage restrictor (42) has in the rest condition a line shaped collapsed damper lumen, then in the infusion condition the deformable flow passage restrictor (42) has a lumen section having a substantially elliptical shape.

In a 95^{th} aspect according to any one of the preceding aspects from 89 to 94, if the deformable flow passage restrictor (42) has in the rest condition a dot shaped collapsed damper lumen (44), then in the infusion condition the deformable flow passage restrictor (42) has a lumen section having a substantially circular shape.

This may be obtained with a very deformable and elastic material.

In a 96^{th} aspect according to any one of the preceding aspects from 89 to 95, said deformable flow passage restrictor (42) is a tube tract, optionally a tube tract of the infusion line (51).

In a 96^{th} bis aspect according to the preceding aspect, the tube tract of the deformable flow passage restrictor (42) has a flatten shape, in particular wherein said tube tract has a section, orthogonal to the fluid direction, having a substantially external elliptic and/or rectangular shape.

In a 96^{th} ter aspect according to any one the preceding aspects 96 and 96 bis, said tube tract extends along an infusion direction (ID) by a length comprised between 5 mm and 100 mm, in particular between 10 mm and 50 mm, optionally between 10 mm and 30 mm.

In a 96^{th} quater aspect according to any one the preceding aspects 96, 96 bis and 96 ter, said tube tract defines internally the damper lumen (44) of the deformable flow passage restrictor (42), said damper lumen (44) having a substantially constant cross section.

In a 97^{th} aspect according to any one of the preceding aspects from 89 to 96, the deformable flow passage restrictor (42) is a tube tract of the infusion line (51), and wherein:
a tube wall of the deformable flow passage restrictor (42) has a thickness substantially equal to a thickness of a tube wall of the rest of the infusion line, in particular substantially equal to a thickness of an upstream tube wall of the infusion line arranged upstream of the deformable flow passage restrictor (42),
in particular wherein the deformable flow passage restrictor (42) defines, at least in the rest condition, a flat tube tract, optionally wherein the deformable flow passage restrictor (42) is made of the same material of the rest of the infusion line;
   or
a tube wall of the deformable flow passage restrictor (42) has a thickness greater than a thickness of a tube wall of the rest of the infusion line, in particular greater than a thickness of an upstream tube wall of the infusion line positioned just upstream the deformable flow passage restrictor (42),
optionally wherein the deformable flow passage restrictor (42) is made of the same material of the rest of
the infusion line, in particular one of silicone, silicone-based material, latex or rubber.

In a 98^{th} aspect according to any one of the preceding aspects from 89 to 97, the deformable flow passage restrictor (42) is made as a single piece.

In a 99^{th} aspect according to any one of the preceding aspects from 89 to 98, the deformable flow passage restrictor (42) and the infusion line (51) are made as a seamless single piece.

In a 99^{th} bis aspect according to any one of the preceding aspects from 89 to 98, the deformable flow passage restrictor (42) and the infusion line (51) define in combination a seamless single piece.

In a 100^{th} aspect according to any one of the preceding aspects from 89 to 99, at least in the rest condition, a lumen of a tube tract of the infusion line (51) upstream the deformable flow passage restrictor (42) reduces gradually in size towards the damper lumen (44) of the deformable flow passage restrictor (42) defining an narrowing lumen section (43).

In a 101^{st} aspect according to the preceding aspect, the narrowing lumen section (43) defines an angle, between the section of the upstream tube tract and the damper section of the deformable flow passage restrictor (42), comprised between 10° and 45°.

In a 102^{nd} aspect according to any one of the preceding aspects from 100 to 101, the narrowing lumen section (43) extends in length along the flow direction by a length of at least 2 mm, in particular at least 5 mm, optionally between 2 mm and 20 mm.

In a 103^{rd} aspect according to any one of the preceding aspects from 100 to 102, the narrowing lumen section (43) extends in length along a curvilinear or straight line path.

In a 104^{th} aspect according to any one of the preceding aspects from 89 to 103, the deformable flow passage restrictor (42) remains in the rest condition if a pressure in the infusion line (51) upstream and downstream the deformable flow passage restrictor (42) is below the atmospheric pressure.

In a 105^{th} aspect according to any one of the preceding aspects from 89 to 104, the deformable flow passage restrictor (42) is configured to switch from the rest condition to the infusion condition if a pressure in the infusion line (51) is higher than the atmospheric pressure.

In a 106^{th} aspect according to any one of the preceding aspects from 89 to 105, the pressure damper (40) comprises a squeezing device (90) operating in thrust on the deformable flow passage restrictor (42), in particular on the tube tract of the deformable flow passage restrictor (42).

In a 107^{th} aspect according to the preceding aspect, the squeezing device (90) is configured to:
provide a closing force contribution to keep the damper lumen (44) substantially closed in the rest condition, and
allow the deformable flow passage restrictor (42) to switch in the infusion condition when a pressure in the infusion line upstream the pressure damper overcomes a predefine threshold, said predefine threshold being in particular at least 1.1 atmosphere.

In a 108^{th} aspect according to any one of the preceding aspects from 106 to 107, the squeezing device (90) is arranged around the deformable flow passage restrictor (42) and acts in compression on an external surface of the deformable flow passage restrictor (42).

In a 109^{th} aspect according to any one of the preceding aspects from 106, the squeezing device (90) comprises an elastic element (91), optionally a spring, such as a spiral spring or a flat spring or a spring-like element, acting in thrust on the external surface of the deformable flow passage restrictor (42).

In a 110^{th} aspect according to the preceding aspect, the elastic element (91) of the squeezing device (90) is preloaded in the rest condition.

In a 111^{th} aspect according to the preceding aspect, the preload of the elastic element (91) is adjustable, for example manually by an operator.

In a 112^{th} aspect according to any one of the preceding aspects from 110 to 111, the elastic element (91) of the squeezing device (90) is adjustable in thrust.

In a 113^{th} aspect according to any one of the preceding aspects from 106 to 112, the squeezing device (90) comprises respective one or more plates (92) acting in thrust on the external surface of the deformable flow passage restrictor (42) in particular along a length greater than 1 cm, in particular greater than 2 cm, in particular between 1 cm and 5 cm or between 1 cm and 3 cm.

In a 114^{th} aspect according to any one of the preceding aspects, the infusion pump (54) is configured to generate an head pressure greater than a predefined threshold to open either the one-way valve (46; 75) or the deformable flow passage restrictor (42).

In a 115^{th} aspect according to any one of the preceding aspects, the infusion pump (54) is configured to generate an overpressure between upstream and downstream the deformable flow passage restrictor (42) greater than 0,05 Bar or 0,1 Bar, optionally greater than 0,2 Bar or 0,3 Bar.

In a 116^{th} aspect according to any one of the preceding aspects, the infusion pump (54) comprises an occlusive pump, in particular a peristaltic pump.

### DRAWINGS

Some embodiments and some aspects of the invention will be described below with reference to the attached drawings, provided for illustrative purposes only, wherein:

FIGS. 1 and 2 are schematic views of possible different embodiments of an extracorporeal blood treatment apparatus comprising the disposable set according to the present invention;

FIG. 3 is a section view of a fluid connector housing a one-way valve according to an embodiment of the disposable set of the present invention;

FIGS. 4-6 are a schematic views of a flow passage restrictor according to different embodiments of the disposable set of the present invention;

FIG. 7 is a schematic view of a disposable set according to the present invention fluidly connected to a (schematic) ECMO circuit;

FIG. 8A is a lateral view of a duckbill valve according to the present invention;

FIG. 8B shows the duckbill valve of FIG. 8A rotated 90° about the fluid axis F;

FIG. 8C is a section view of the duckbill valve of FIG. 8B;

FIG. 9 is a section view, along a fluid axis, of a deformable flow passage restrictor according to the present invention in a rest condition;

FIG. 9A is a section view, orthogonal to a fluid passage axis, of the deformable flow passage restrictor of FIG. 9 wherein the damper lumen is collapsed defining a line;

FIG. 9A″ is a section view, orthogonal to a fluid passage axis, according to an alternative embodiment of the deformable flow passage restrictor of FIG. 9 wherein the damper lumen is collapsed defining a dot;

FIG. 9B is a section view, orthogonal to a fluid passage axis, of the infusion line upstream and/or downstream the deformable flow passage restrictor of FIG. 9;

FIG. 10 is a section view, along a fluid axis, of a deformable flow passage restrictor according to the present invention in an infusion condition;

FIG. 10A is a section view, orthogonal to a fluid passage axis, of the deformable flow passage restrictor of FIG. 10 wherein the damper lumen is opened defining an elliptical shape;

FIG. 10A″ is a section view, orthogonal to a fluid passage axis, according to an alternative embodiment of the deformable flow passage restrictor of FIG. 10 wherein the damper lumen is opened defining a circular shape;

FIG. 10B is a section view, orthogonal to a fluid passage axis, of the infusion line upstream and/or downstream the deformable flow passage restrictor of FIG. 10;

FIG. 11 is a section view, along a fluid axis, of a deformable flow passage restrictor according to a further embodiment of the present invention in a rest condition;

FIG. 11A is a section view, orthogonal to a fluid passage axis, of the deformable flow passage restrictor of FIG. 11 wherein the damper lumen is collapsed defining a line;

FIG. 11A″ is a section view, orthogonal to a fluid passage axis, according to an alternative embodiment of the deformable flow passage restrictor of FIG. 11 wherein the damper lumen is collapsed defining a dot;

FIG. 11B is a section view, orthogonal to a fluid passage axis, of the infusion line upstream and/or downstream the deformable flow passage restrictor of FIG. 11;

FIG. 12 is a section view, along a fluid axis, of a deformable flow passage restrictor according to the present invention in an infusion condition;

FIG. 12A is a section view, orthogonal to a fluid passage axis, of the deformable flow passage restrictor of FIG. 12 wherein the damper lumen is opened defining an elliptical shape;

FIG. 12A″ is a section view, orthogonal to a fluid passage axis, according to an alternative embodiment of the deformable flow passage restrictor of FIG. 12 wherein the damper lumen is opened defining a circular shape;

FIG. 12B is a section view, orthogonal to a fluid passage axis, of the infusion line upstream and/or downstream the deformable flow passage restrictor of FIG. 12;

FIG. 13 is a section view, along a fluid axis, of a deformable flow passage restrictor according to another embodiment of the present invention in a rest condition, and wherein the pressure damper comprises a squeezing device;

FIG. 13A is a section view, orthogonal to a fluid passage axis, of the deformable flow passage restrictor of FIG. 13 wherein the damper lumen is collapsed defining a line;

FIG. 13B is a section view, orthogonal to a fluid passage axis, of the infusion line upstream and/or downstream the deformable flow passage restrictor of FIG. 13;

FIG. 14 is a section view, along a fluid axis, of a deformable flow passage restrictor according to the present invention in an infusion condition, and wherein the pressure damper comprises the squeezing device;

FIG. 14A is a section view, orthogonal to a fluid passage axis, of the deformable flow passage restrictor of FIG. 14 wherein the damper lumen is opened defining an elliptical shape;

FIG. 14B is a section view, orthogonal to a fluid passage axis, of the infusion line upstream and/or downstream the deformable flow passage restrictor of FIG. 14.

### DEFINITIONS

In this detailed description, corresponding parts illustrated in the various figures are indicated with the same numerical references. The figures may illustrate the invention through non-scale representations; therefore, parts and components illustrated in the figures relating to the object of the invention may relate exclusively to schematic representations.

The terms “upstream” and “downstream” refer to a direction or trajectory of advancement of a fluid configured to flow within the connector or along the fluid line or duct during normal usage of the apparatus, for example during an extracorporeal blood treatment. During normal use of the apparatus the blood pump pumps blood from the patient vascular access along the blood withdrawal line, cross the filtration unit and back to the patient along the blood return line. Infusion fluids are infused from the respective fluid sources towards the blood circuit and into the blood. Dialysis fluid (if any) flows from the dialysis line to the filtration unit and towards the effluent line. Blood flow and dialysis flow are countercurrent in the filtration unit.

In an ECMO circuit, the blood direction is defined by the respective blood pump from the blood withdrawal duct towards the blood return duct.

We define the “dialysis fluid” as the treatment fluid introduced to the second chamber of the filtration unit 2. The dialysis fluid may be on-line prepared or pre-packaged in sterile bags. Usually in CRRT apparatuses/applications the dialysis fluid, but also the replacement fluids (possibly also regional anticoagulant fluid and/or ion re-establishing solution fluid) are contained in (disposable) bags.

We define the “dialysate” or “effluent” as the fluid from the outlet from the second chamber of the filtration unit 2. Dialysate or effluent is the spent dialysis fluid, comprising the uremic toxins removed from the blood and may include ultrafiltrate fluid.

We define “regional anticoagulant” as a substance which, once mixed with extracorporeal blood, substantially prevents blood coagulation in the extracorporeal blood circuit and which is quickly metabolized by the patient, thus avoiding systemic anticoagulation.

We define the term “degassing” as a process wherein gases dissolved in a fluid, such as an infusion fluid or blood, tend to get free due to a local low pressure or due to fluid warming, which leads to separation of the gases from the liquid phase of the fluid, consequently generating bubbles into the fluid.

We define “negative pressure” a pressure below the local atmospheric pressure.

We define “positive pressure” a pressure above the local atmospheric pressure.

### DETAILED DESCRIPTION

### Disposable Set 100

Reference number 100 is directed to a disposable set for an extracorporeal blood treatment apparatus, such as a hemodialysis apparatus for performing a haemodialysis treatment (HD), an ultrafiltration apparatus for performing an ultrafiltration treatment (UF), a haemofiltration apparatus for performing a haemofiltration treatment (HF) or a haemodiafiltration apparatus for performing a haemodiafiltration treatment (HDF).

Alternatively, the disposable set 100 may be directed to perform an extracorporeal blood treatment such as a Therapeutic Plasma Exchange (TPE) treatment. The TPE treatment is a procedure wherein the patient's blood passes through an apheresis machine for plasma filtration and removal: plasma is then replaced by a replacement fluid, such as a plasma from a donor, albumin, or saline.

Alternatively, the disposable set 100 may be directed to perform an extracorporeal blood treatment such as a HemoPerfusion treatment for blood purification: in particular HemoPerfusion treatment consists of the passage of the patient's blood through a device, usually a column, which contains adsorbent particles configured to remove toxins from blood, i.e. in case of treatment of poisoning.

Alternatively, the disposable set 100 may be directed to perform an extracorporeal blood treatment for CO₂ removal treatment from the blood: the CO₂ removal treatment may be performed during a dialysis treatment, or may be performed by itself through a blood circuit ad hoc.

Furthermore, the disposable set 100 may be connected to a further extracorporeal blood treatment apparatus, such as an apparatus for blood oxygenation, namely an ExtraCorporeal Membrane Oxygenation ECMO treatment apparatus.

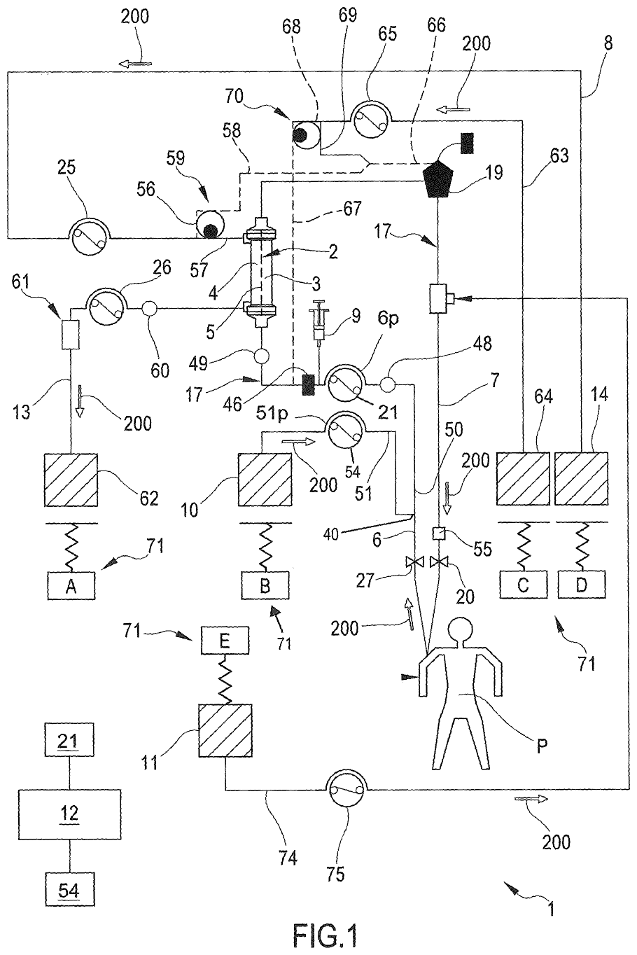

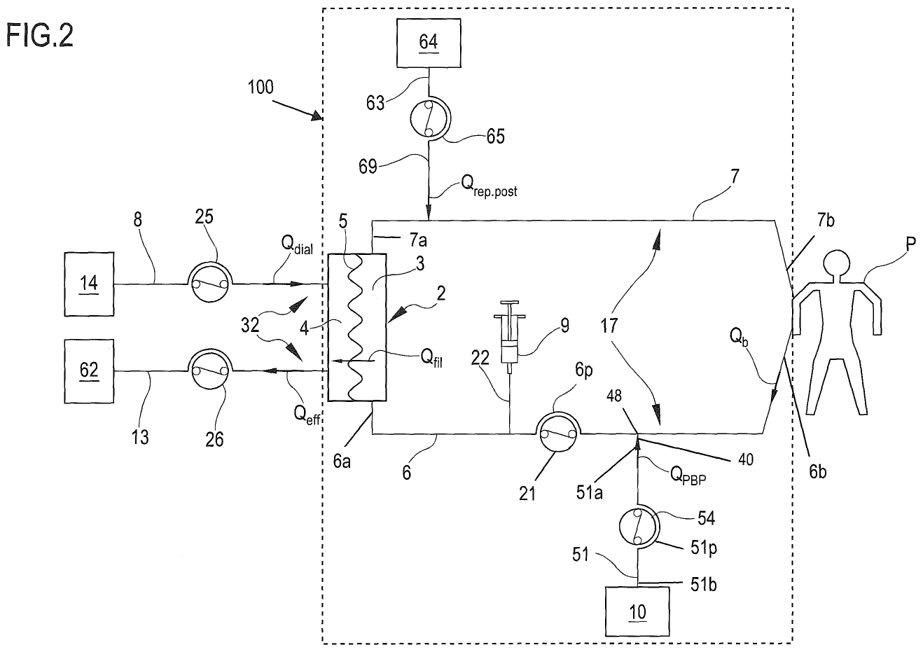

According to the medical fields listed above, the disposable set 100, schematically shown in FIGS. 1 and 2, comprises blood and fluid lines configured to be associated to the respective treatment apparatus 1, for example to peristaltic pumps to promote fluid flow and respective sensors and actuators to operate the circuit. Here after a detailed description of the disposable set is provided. In this regard, the pumps are not part of the disposable set 100, even if for sake of simplicity, the dot lines in FIG. 2 encompass also pumps (e.g., infusion pump 54, blood pump 21 and post-infusion pump). Syringe pump 9 and bags 10 and 64 may be part or may be not part of the disposable set depending on its configuration.

The disposable set 100 comprises at least one filtration unit 2, which is configured to treat the blood withdrawn from the patient. The filtration unit 2 may be a filter for performing one between a haemodialysis treatment (HD), an ultrafiltration treatment (UF), a haemofiltration treatment (HF) and a haemodiafiltration treatment (HDF). The filtration unit 2 may alternatively be an absorber unit, or sorbent cartridge for a sorbent system in case of hemoperfusion treatments.

In an embodiment, the filtration unit 2 has a primary chamber 3 and a secondary chamber 4 separated by a semi-permeable membrane 5, wherein the primary chamber 3 receives the blood withdrawn from the patient, while the secondary chamber 4 receives waste products and fluid removed from the blood and discharges it through an outlet connected to an effluent fluid line 13. Depending upon the treatment, the membrane of the filtration unit may be selected to have different properties and performances. According to a further embodiment, the secondary chamber of the filtration unit 2 further comprises, in addition to the outlet, an inlet configured to receive fluid, i.e. dialysis fluid, from a dialysis liquid supply line 8.

The disposable set may comprise the effluent fluid line 13 connected to the outlet of the dialyzer 2: the effluent fluid line 13 may also comprise a respective effluent pump tract configured to be engaged to a dialysate pump 26.

Analogously, in the case wherein the dialyzer comprises an inlet, the disposable set may comprise a supply line 8 connected to the inlet of the dialyzer and configured to deliver dialysis fluid to the dialyzer 2. The dialysis liquid supply line 8 also comprises a respective supply pump tract configured to be engaged to a dialysis fluid pump 25 of the extracorporeal blood treatment apparatus.

The blood circuit further comprises a blood withdrawal line 6 extending between a first end 6a connected to the filtration unit 2 and a second end for connection to a patient P. In case the filtration unit 2 comprises the primary and secondary chambers, the blood withdrawal line 6 extends between a first end 6a connected to the inlet of the primary chamber 3 of the filtration unit 2 and a second end 6b for connection to a patient P. The blood withdrawal line 6 is configured to receive blood from the patient P and carry the blood along a withdrawn direction 200 from the second end 6b to the first end 6a of the blood withdrawal line 6.

The blood circuit 17 further comprises a blood return line 7 extending between a first end 7a connected to the filtration unit 2 and a second end 7b for connection to said patient P. In case the filtration unit 2 comprises the primary and secondary chambers, the blood return line 7 extends between a first end 7a connected to the outlet of the primary chamber 3 of the filtration unit 2 and a second end 7b for connection to a patient P. The blood return line 7 is configured to receive blood from the outlet of the filtration unit 2 and carry the blood along a return direction defined from the first end to the second end of the blood return line 7.

The withdrawal line 6 and the return line 7 may be connected to the blood stream of the patient through a vascular access, through a needle, a catheter, or an access device.

The withdrawal line 6 and the return line 7 may be made of a flexible material, for example PVC or other plastic based bio compatible material: the blood lines 6, 7 may also be transparent to allow an operator to see the blood flowing within the lines.

The blood withdrawal line 6 of the blood circuit may have a fluid passage section comprised between 3 mm² and 20 mm² corresponding to inner diameters between 2 mm and 5 mm (the more common inner diameters are included between 2.5 mm and 4.5 mm).

The blood withdrawal line 6 includes a pump tract 6p configured to be engaged to a blood pump 21 of the extracorporeal blood treatment apparatus which is configured to generate a blood flow and the blood circulates in the blood circuit in a direction 200 from the blood withdrawal line 6 towards the filtration unit 2. At least during an operating condition of the apparatus, usually a positive pressure regimen is experienced downstream the blood pump tract 6p, and an access negative pressure regimen is experienced upstream the blood pump tract 6p. Notably the access negative pressure is lower than the atmospheric pressure.

In one example, the blood-pump 21 may be implemented by a pump-rotor element integrated with the dialyzer and operably connected to a magnetic field for its operation.

The blood pump tract 6p may be a portion of the blood withdrawal line 6 itself, which is interposed between the first end 6a and the second end 6b of the blood withdrawal line 6.

Thus, the blood withdrawal line 6 comprises:
the blood pump tract 6p extending between the first end 6a and the second end 6b,
a first tract extending between the first end 6a and the blood pump tract 6p,
a second tract extending between the second end 6b of the blood withdrawal line 6 and the blood pump tract 6p.

The blood pump tract 6p may be different with respect to the first and the second tracts of the blood withdrawal line 6 in terms of dimension and/or material. In particular the blood pump tract 6p may have an external dimension, i.e. an external diameter, bigger than an external dimension, i.e. an external diameter, of the first and/or second tracts of the blood withdrawal line. In addition or alternatively, the blood pump tract 6p may have an internal dimension, i.e. an internal diameter, defining the fluid passage of the blood pump tract 6p, which is bigger than an internal dimension, i.e. an internal diameter, of the first and/or second tracts of the blood withdrawal line. The blood pump tract 6p may be a different tube segment with respect to the first and the second tracts, wherein the blood pump tract 6p is engaged/coupled to the first and the second tracts by a gluing or welding step performed during a manufacturing process of the disposable set 100. Usually, the blood withdrawal line 6 has uniform section with the exclusion of the pump tract 6p that may have a slightly bigger inner section, e.g., 6-8 mm.

The blood pump tract 6p may also has a stiffness/elasticity different from a stiffness of the first and the second tracts of the blood withdrawal line 6: for example the blood pump tract 6p may be more flexible or elastic than the first and the second tracts of the blood withdrawal line 6. The blood pump tract 6p, in order to withstand the fatigue stresses caused by the peristaltic pump 21 of the external blood treatment apparatus, is made by a more flexible/elastic material with respect to a material of the first and/or second tracts of the blood withdrawal line 6. The blood circuit further comprises an infusion line 51 extending between a first end 51a connected to the blood withdrawal line 6 upstream the blood pump tract 6p at a fluid access, and a second end 51b for connection to an infusion substance source: the blood pump tract 6p is interposed between the filtration unit 2 and the first end 51a of the infusion line 51.

The blood withdrawal line 6 and the infusion line 51 may be made by the same material, i.e. PVC, silicone, or other plastic based material. The blood withdrawal line 6 and the infusion line 51 may also have the same geometry, for example a circular cross section having constant internal diameter and constant external diameter. Notably, both the blood withdrawal line 6 and the infusion line 51 are flexible.

The infusion line 51 may have a fluid passage section comprised between 0.7 mm² and 20 mm² or the fluid passage section may have a diameter comprised between 1 mm and 5 mm.

The infusion substance source 10, connected or configured to be connected to the infusion line 51, may be an infusion bag. The bag may house a fluid infusion solution comprising one between bicarbonate, acetate, lactate, citrate, a replacement fluid, saline, and a regional anticoagulant solution. Notably, the disposable set may comprise the bag, which is connected to the second end 51b of the infusion line 51.

The infusion line 51 may further comprise a respective infusion pump tract 51p interposed between the first and 51a the second end 51b of the infusion line 51: the infusion pump tract 51p of the infusion line 51 is configured to be engaged by an infusion pump 54, i.e. a peristaltic pump, configured to determine, at least during an operating condition of the infusion pump 54, a positive pressure downstream the infusion pump tract 51p to allow infusion fluid to flow in a direction from the second to the first end of the infusion line 51 towards the blood withdrawal line 6. The infusion pump tract 51p may comprise the same features previously described according to the blood pump tract 6p of the blood withdrawal line 6.

Thus the infusion line 51 comprises:
the infusion pump tract 51p extending between the first end 51a and a second end 51b,
a first tract extending between the first end of the infusion line 51 and the first end of the infusion pump 54 tract,
a second tract extending between the second end of the infusion line 51 and the second end of the infusion pump 54 tract,
   wherein the infusion pump tract is different from the first and the second tracts of the infusion line 51 in terms of dimensions and/or material. In particular the infusion pump tract may have an external dimension, i.e. an external diameter, bigger than an external dimension, i.e. an external diameter, of the first and/or second tracts of the infusion line 51. In addition or alternatively, the infusion pump tract may have an internal dimension, i.e. an internal diameter, defining the fluid passage which is bigger than an internal dimension, i.e. an internal diameter, of the first and/or second tracts of the infusion line 51. The infusion pump tract 51p may be a different tube segment with respect to the first and the second tracts, wherein the infusion pump tract is engaged/coupled to the first and the second tracts by a gluing or welding step performed during a manufacturing process of the disposable set 100.

The infusion pump tract 51p may also has a stiffness/elasticity different with respect to a stiffness of the first and the second tracts of the infusion line 51: for example the infusion pump tract 51p may be more flexible/elastic than the first and the second tracts of the infusion line 51. The infusion pump tract, in order to withstand the fatigue stresses caused by the peristaltic infusion pump 54 of the external blood treatment apparatus, is made by a more flexible/elastic material with respect to a material of the first and/or second tracts of the infusion line 51.

The blood circuit 17 comprises an intersection portion where the infusion line 51 is joined to the blood withdrawal line 6 to allow the infusion fluid, flowing within the infusion line 51, to be infused into the blood withdrawal line: in particular the intersection portion includes an infusion connector 48 having a connector body defining an internal volume.

The infusion connector comprises a blood inlet 48a and a blood outlet 48b fluidly communicating each other and with the internal volume of the connector, and wherein the blood inlet 48a is connected to the blood withdrawal line 6 and faces the second end 6b of the blood withdrawal line 6, while the blood outlet 48b is connected to the blood withdrawal line 6 and faces the blood pump tract 6p of the blood withdrawal line 6. In particular the blood flows within the infusion connector in the withdrawal direction 200 from the blood inlet 48a to the blood outlet 48b of the infusion connector 48. The infusion connector 48 further comprises an infusion inlet 48c fluidly communicating with the internal volume of the connector body and connected to the first end 51a of the infusion line 51. Thus, the infusion inlet 48c, the blood inlet 48a and the blood outlet 48b are in fluid communication each other defining a three-way connector having two inlets and one outlet.

The infusion connector 48 may be made by plastic material: in particular the infusion connector is generally stiffer than the infusion line 51 and/or of the blood withdrawal line 6. On the contrary, the infusion line 51 is flexible, in particular more flexible than the infusion connector 48. Analogously, the blood withdrawal line 6 is more flexible than the infusion connector: for example, the blood withdrawal line 6 may be as flexible as the infusion line 51.

The infusion connector 48 may be in one piece with the blood withdrawal line 6 and with the infusion line 51: in this case, the first end of the infusion line 51 is welded or glued to the infusion inlet of the infusion connector. Also the blood withdrawal line 6 is welded or glued to the blood inlet and to the blood outlet of the infusion connector 48. The term “welded” may refer to thermal or chemical welding between the blood or infusion line 51 and the infusion connector. Thus the infusion connector 48, the blood withdrawal line 6 and the infusion line 51 may define a non-separable fluid line set. In particular the disposable set including the blood withdrawal line 6, the infusion line 51, the blood return line 7 and the dialyzer 2, may be in one piece, defining thereby a one piece disposable set 100. In particular no removable connectors may be provided in the disposable set for connection of the blood circuit to the infusion line 51 and the filter unit 2.

Notably the infusion fluid may be for example a replacement fluid or a saline or a regional anticoagulation fluid, depending on the specific treatment and/or on the specific step of an apparatus working sequence (e.g., priming vs patient blood treatment). The infusion fluid may include a buffer (e.g., bicarbonate, acetate or lactate), one or more electrolytes (e.g., sodium, magnesium, calcium, potassium, etc.), or a regional anticoagulant, such as citrate (e.g., trisodium citrate or citric acid).

According to one embodiment, the infusion line 51 is connected to the infusion connector 48 at a coupling portion defining a discontinuity-free coupling in the flow passage. In other terms, an internal flow passage in substantially constant from the infusion line 51 up to the infusion connector, including the coupling portion: de facto, the coupling portion does not define a discontinuity in the internal flow passage, in order to avoid turbulences in the fluid flow.

Notably, the negative pressure regimen in the blood line of the disposable set extends at least between the blood pump tract 6p of the blood withdrawal line 6 and the intersection portion with the infusion line 51: in particular the negative pressure regimen may also extend at least between the blood pump tract 6p of the blood withdrawal line 6 and the second end 6b of the blood withdrawal line 6 at the connection with the patient.

The disposable set 100 further comprises a pressure damper 40 arranged towards or at the first end of the infusion line 51: in particular the pressure damper 40 may be arranged at the infusion connector, namely at the intersection between the blood withdrawal line 6 and the infusion line 51, or on the infusion line 51 in proximity of the intersection, i.e. at a distance from the intersection comprised between 0.1 cm and 6 cm, more in particular between 0.2 and 3 cm. The pressure damper 40 is arranged on the infusion line 51 between the infusion pump tract 51p and the blood withdrawal line 6.

The pressure damper 40 is configured to prevent, or to reduce an amount of, the access negative pressure to extend in the infusion line 51 upstream the pressure damper 40 with respect to the direction of the infusion fluid: the direction of the infusion fluid is directed from the second end 51b to the first end 51a of the infusion line 51. As already mentioned, the access negative pressure may cause degassing especially in the infusion fluid, thereby generating bubbles, which are problematic when present in the blood circuit for all the above explained reasons. Thus, the pressure damper 40 allows the infusion fluid in the infusion line 51 to be, during an operating condition of the blood treatment apparatus, at a pressure higher than the access negative pressure, thereby reducing or preventing degassing of the infusion fluid.

In order to achieve the task of reducing or preventing the negative pressure in the infusion line, two different approaches are provided. In a first approach, a suitable restriction of the flow passage in the infusion line is obtained, but without preventing the fluid flow (i.e., the fluid passage, though restricted, is always open independent on the apparatus working condition). In a second approach a suitable one-way valve is used with an opening threshold so to prevent fluid passage unless a certain pressure differential over the one way valve is present in the infusion line.

According to the first embodiment, the pressure damper 40 may be a flow passage restrictor 41 which comprises a damper passage section configured to allow the infusion fluid to pass through: the damper passage section is smaller than the fluid passage section of the infusion fluid: for example the damper passage section may be at least 50% smaller than the fluid passage section, in particular at least 80% smaller, in particular more than 90% smaller. The flow restrictor may define a fluid passage section having circular shape.

The flow passage restrictor 41 of the pressure damper 40 may comprise a septum, e.g., having circular shape, having a fluid passage which reduces the fluid passage section with respect to the passage section of the infusion line 51. In order to achieve a sufficient pressure drop considering a typical infusion rate of about 1000 ml/h, the septum should define a fluid passage having a cylindrical section with a diameter less than 0.4 mm in case a fluid passage length lower than 60 mm is desired. Though it may be difficult to mold a plastic element with an inner diameter of 0.5 mm or less, a metallic portion may be eventually used. Needles such as 33 G (0.24 mm), 32 G (0.26 mm), 30 G (0.3 mm), 27 G (0.41 mm) or 26 G (0.45 mm) may be used. Portions of the required length may be indeed obtained.

The fluid passage axially extends along a flow direction of the infusion line 51 by a fluid passage length comprised between 1 mm and 30 mm, in particular between 2 mm and 15 mm, in particular between 4 mm and 10 mm. According to the embodiment comprising the septum, the flow passage restrictor 41 of the pressure damper 40 may define a sharp discontinuity for the fluid flow in the infusion line 51.

According to an embodiment, the damper passage section of the flow passage restrictor 41 is substantially constant along the fluid passage length. Notably, the flow passage restrictor 41 defines an aperture, which puts the infusion line 51 in fluid communication with the blood withdrawal line 6. The aperture is fixed in size: de facto the latter cannot be modified by an operator during the treatment.

According to an embodiment, the flow restrictor of the pressure damper 40 may be arranged into the infusion connector, in particular inside the infusion inlet 48c of the infusion connector 48.

Alternatively, the flow restrictor of the pressure damper 40 may be arranged on the infusion line 51 upstream, with respect to the flow direction of the infusion fluid, the infusion connector 48 and as close as possible to the blood withdrawn line 6. For example, the pressure damper 40 may be a damper connector interposed between an upstream and a downstream tract of the infusion line 51, wherein the flow restrictor is arranged within said damper connector: this damper connector may extends along the infusion fluid direction by a length comprised between 5 mm and 30 mm, in particular between 10 and 20 mm. According to this embodiment, the damper connector may be stiffer than the infusion line 51 and made of a material different and stiffer than the material of the infusion line 51, for example the damper connector may be made of metal and the infusion line 51 of flexible PVC or silicone.

Notably, when a passage restrictor is provided, the infusion line 51 defines an internal flow passage having a fluid passage section substantially constant up to the passage restrictor of the pressure damper 40: thus the passage restrictor defines a discontinuity in the fluid passage section to prevent the negative pressure to extend into the infusion line 51.

Indeed, the flow passage restrictor 41 is configured to define a pressure drop in the infusion fluid during an operating condition of the treatment apparatus, in particular when an infusion fluid flows within the infusion line 51 towards the blood withdrawal line 6. The pressure drop is considered as a differential pressure between the infusion line and the blood withdrawal line upstream the blood pump tract: in particular the pressure drop is considered as a differential pressure between a section just upstream the pressure damper 40, and a section just downstream the pressure damper 40.

FIG. 4 shows a further embodiment wherein the flow passage restrictor 41 comprises a tube tract defining the first end of the infusion line: the tube tract has an internal diameter lower than the internal diameter of the infusion line and extends by a length higher than the passage restrictors described in the preceding embodiments. In particular the damper passage of the tube tract extends by a length comprised between 30 mm and 200 mm, in particular between 40 mm and 160 mm, more in particular between 55 mm and 150 mm, and the damper passage section extends by a diameter comprised between 0.3 mm and 0.6 mm, in particular between 0.35 mm and 0.55 mm.

More in detail, and according to a specific embodiment, if the damper passage section has a diameter substantially equal to 0.5 mm, the damper passage extends by a length substantially equal to 145 mm optionally ±15 mm, while if the damper passage section has a diameter substantially equal to 0.4 mm, the damper passage extends by a length substantially equal to 60 mm optionally ±10 mm. The diameter of the damper passage is substantially constant over the length.

Notably the tube tract defining the flow passage restrictor 41 may be made of the same material of the rest of the infusion line, i.e. a flexible material, such as medical grade PVC, silicone, or of another bio compatible material.

In order to avoid degassing of the infusion fluid, the internal diameter of the flow passage restrictor 41 depends on the length of the flow passage restrictor 41 and vice versa: indeed, given the properties of the infusion fluid and the flow rate set for the infusion fluid, the pressure drop across the pressure damper is defined by the damper passage section and by the length of flow passage restrictor 41. In particular the damper passage section is proportional to the length of the flow passage restrictor 41: in other terms, the smaller is the damper passage section, the lower is the length of the flow passage restrictor 41 needed to obtain a sufficient pressure drop to avoid degassing of the infusion fluid in the infusion line 51.

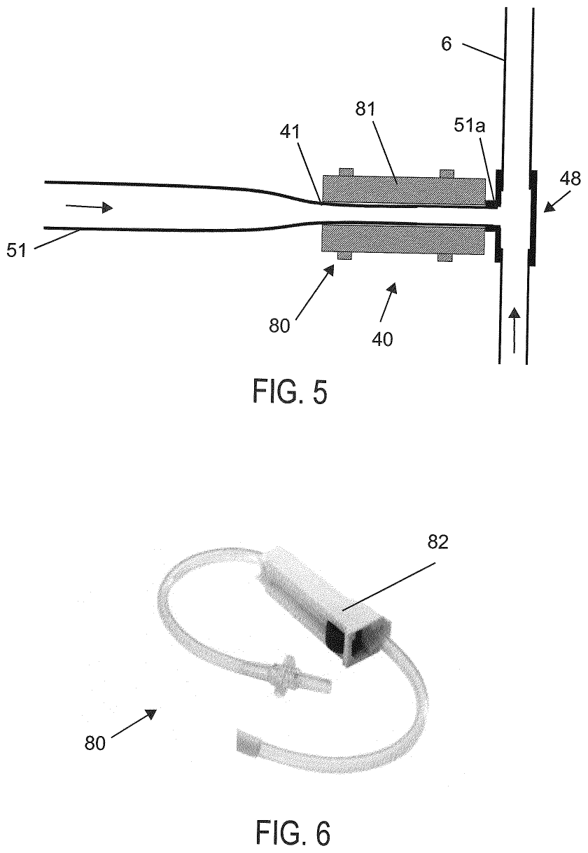

Still according to a further embodiment, the flow passage restrictor 41 may be defined by a clamp 80 configured to clamp the infusion line 51 and to deform the infusion line so that the internal lumen is reduced (but not completely closed as normal clamps do). Thus the clamp acts on the external surface the infusion line 51, radially compressing the line and reducing the internal damper passage section. The clamp 80 may comprise a portable clamp 81, as schematically shown in FIG. 5, comprising a clamping seat configured to receive a portion of the infusion line 51 and having a fixed size to determine a predefined lumen reduction of the infusion line 51 along a certain tube length: this type of clamp is designed according to a specific geometry, i.e. the size, of the infusion line, in order to cause a predefined lumen restriction. Also in this embodiment, a relation exists between the lumen restriction and the length of the tube portion that is restricted. The longer the tube portion that is restricted, the lower is the lumen restriction and vice versa.

Alternatively, the clamp 80 may comprise a variable clamp 82, as shown in FIG. 6, comprising a clamping seat configured to receive a portion of the infusion line 51 and having a regulator for varying the lumen reduction of the infusion line 51.

Notably, the flow passage restrictor 41 defined by the clamp 80 is defined by the same geometrical features already described according to the previous embodiment, in particular in terms of damper passage section of the flow passage restrictor 41 and the length thereof. In other terms, the clamp has a predefined length and is designed to determine the damper passage section of the flow passage restrictor 41 in order to reduce or avoid degassing of the infusion fluid.

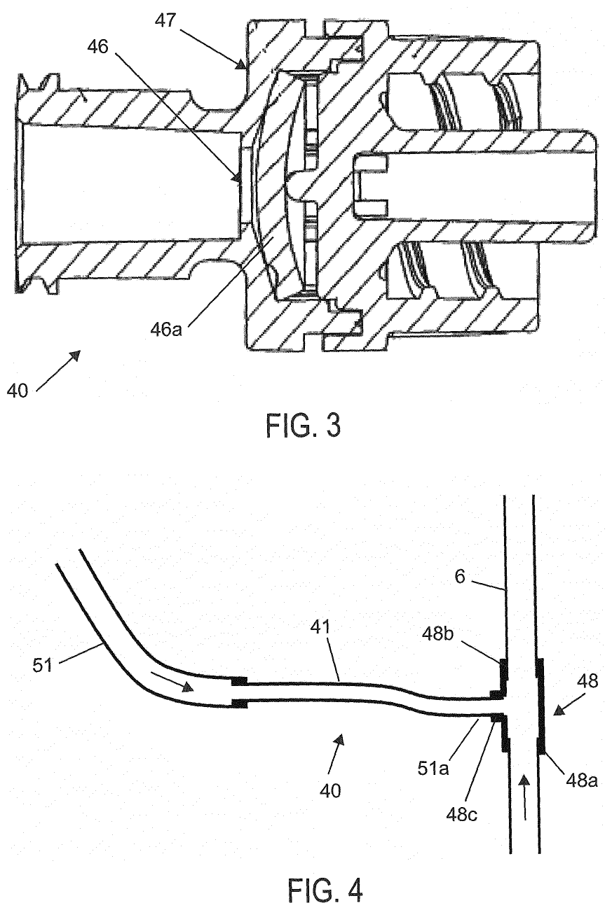

According to a different embodiment, shown in FIG. 3, the pressure damper 40 may comprises a one-way valve 46 configured to allow fluid passage only along the infusion direction directed from the infusion line 51 towards the blood withdrawal line. The one-way valve 46 is configured to move between an open position, in which the fluid passage is allowed in the infusion direction, and a closed position, in which fluid passage is prevented in the infusion direction, in particular in both directions. Notably, the one-way valve only allows fluid flow only in one direction, namely the infusion direction: fluid flow in the opposite direction is always prevented. The one-way valve 46 may be preset at an opening pressure threshold to switch between the open and the closed position and vice versa, so that if a differential pressure between an upstream section and a downstream section of the one-way valve 46 is equal or higher than this opening pressure threshold, the one-way valve 46 is configured to switch to or to maintain the open position. Alternatively, if the differential pressure is lower than this opening pressure threshold, the one-way valve 46 is configured to switch to or to maintain the closed position. Notably, the opening pressure is defined as the differential pressures just upstream and just downstream section the one-way valve: in other terms, the upstream and downstream pressures al local pressure around the internal membrane of the one-way valve.

Notably, the differential pressure is defined between a high pressure at the upstream section of the one-way valve 46, and a low pressure at the downstream section of the one-way valve 46: the high pressure is higher than the low pressure. In particular the high pressure is the pressure in the infusion line 51 upstream, with respect to the infusion direction, to the one-way valve, while the low pressure is the access negative pressure proximal to the catheter/vascular access. The preset opening pressure threshold of the one-way valve 46 corresponds to a differential pressure value comprised between 160 and 500 mmHg, in particular between 190 and 450 mmHg, more in particular between 200 and 400 mmHg, more in detail between 240 and 350 mmHg. These pressure ranges correspond to pressure regimens that normally raise when usual blood flow rates and infusion flow rates are generated within the disposable set. It is clear that the one-way valve opening threshold may be configured to the most common working conditions of the apparatus and, as above discusses, may be optimal for each and every working condition of the apparatus.

The one-way valve 46 may comprise an internal diaphragm 46a movable between the open position and the closed position: the internal diaphragm may be a flexible membrane made of a material between silicone, rubber, PVC or the like. The internal diaphragm is preloaded in the closed position to define the preset opening pressure threshold. In particular the internal diaphragm may have a semispherical shape so that a central portion of the internal diaphragm is axially shifted with respect to the external contour. The geometrical features of the internal diaphragm and the material thereof define the preload, and therefore the preset opening pressure threshold.

According to an embodiment, the disposable set comprises a Luer lock connector 47 housing the one-way valve 46, wherein the internal diaphragm is within an internal fluid passage of the Luer lock connector. The Luer lock connector may be arranged on the infusion line 51, i.e. towards the first end 51a of the infusion line 51, for example at a distance from the first end of the infusion line 51 comprised between 0.1 cm and 6 cm, more in particular between 0.2 and 3 cm.

Alternatively, the one-way valve 46 may be arranged inside the infusion connector 48, in particular inside the infusion inlet 48c of the infusion connector 48.

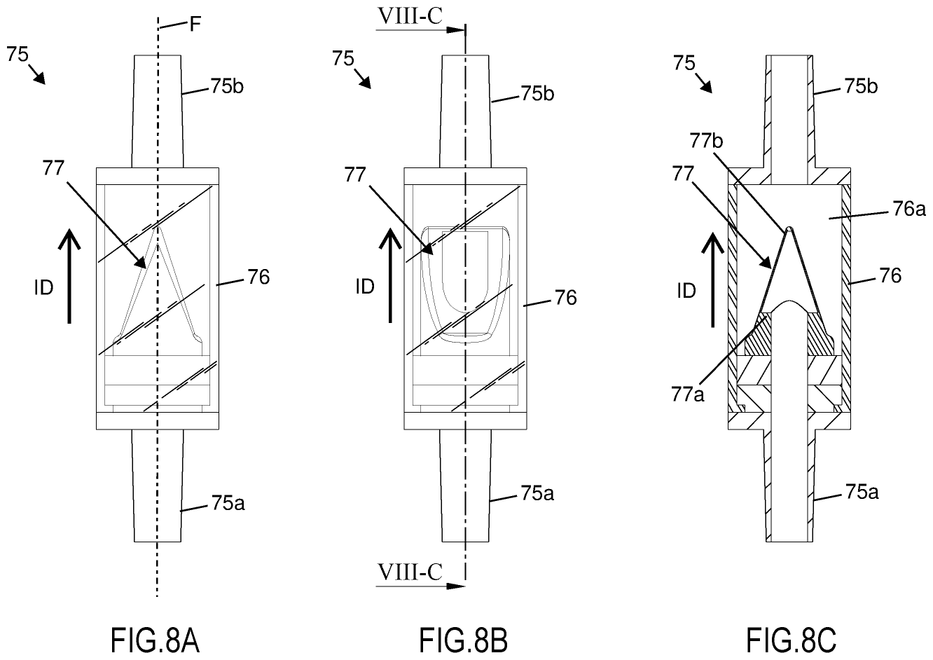

Here after is described a pressure damper 40 comprising a one-way valve according to an embodiment shown in FIGS. 8A and 8B and in the section view of FIG. 8C. The one-way valve of FIG. 8B is rotated by 90° with respect to FIG. 8A about the fluid axis F. The one-way valve is configured to allow fluid passage only in an infusion direction ID directed from the infusion line 51 towards the blood circuit 17, for example towards the withdrawal line 17. The one-way valve may be configured to move between an open position, in which fluid passage is allowed in the infusion direction, and a closed position, in which fluid passage is prevented in the infusion direction and, also, in an opposite direction. The opposite direction is defined as a direction from the blood circuit 17 towards the infusion substance source 10. In other terms, when the one-way valve is in the closed position, the pressure damper 40 defines a fluid tight closure in both directions.

The specific embodiment of FIGS. 8A, 8B, and 8C is directed to a duckbill valve 75, which acts as the one-way valve described above.

The duckbill valve 75 may comprise a housing 76 defining an inner volume 76a: the housing 76 may be made of a rigid material, such as metal or plastic or glass. The material may be selected between materials compatible with the infusion fluid and with an extracorporeal blood treatment apparatus. The housing 76 may be made of a transparent material to allow visualization of the inner volume 76a.

The housing 76 may have a cylindrical shape extending by a length comprised between 2 cm and 10 cm and by a diameter comprised between 1 cm and 5 cm, in particular between 1 cm and 3 cm. The housing 76 may have alternatively other shapes, such as a polygonal section.

The housing 76 may comprises an inlet connector 75a connected or configured to be connected to an upstream tract of the infusion line 51. The inlet connector 75a is configured to receive the infusion fluid from the substance source 10. Thus the upstream tract of the infusion line 51 is interposed between the duckbill valve 75 and the infusion substance source 10. In other terms, the inlet connector 75a of the duckbill valve 75 faces the infusion substance source 10.

The inlet connector 75a has preferably a cylindrical shape to promote engagement to an upstream tract, namely a tube tract, of the infusion line 51. The inlet connector 75a may optionally comprise a Luer Lock connector.

The housing 76 may also comprise an outlet connector 75b connected or configured to be connected to a downstream tract of the infusion line 51. The outlet connector 75b is configured to let the infusion fluid exit from the inner volume 76a of the duckbill valve 75 in a direction towards the blood circuit. The downstream tract of the infusion line 51 is thus interposed between the duckbill valve 75 and the blood circuit 17. The outlet connector 75b has preferably a cylindrical shape to promote engagement to an upstream tract, namely a tube tract, of the infusion line 51. The outlet connector 75b may optionally comprise a Luer Lock connector.

The outlet connector 75b of the duckbill valve 75 faces the blood circuit 17, in particular faces the first end 51a of the infusion line 51.

The housing 76 may further comprise a duckbill component 77 arranged in the inner volume 76a of the housing 76 and configured to selectively define the open position and the closed position of the one-way valve previously described. In more detail, the duckbill component 77 allows the infusion fluid to flow from the inlet connector 75a to the outlet connector 75b and to prevent fluid flow backwards from the outlet connector 75b to the inlet connector 75a.

Notably, the duckbill component 77 is inside the inner volume 76a of the housing 76 and interposed between the inlet connector 75a and the outlet connector 75b, preferably along a fluid path of the infusion fluid. In particular the inlet connector 75a and the outlet connector 75b may be substantially aligned along a line, i.e. a straight line coincident with the infusion fluid passage direction: the duckbill component 77 may be aligned along said line, so that inlet connector 75a, the outlet connector 75b and the duckbill component 77 are aligned along a straight line. Preferably, the inlet connector 75a of the duckbill valve 75 is opposite to the outlet connector 75b of the duckbill valve 75 with respect to the duckbill component 77.

The duckbill component 77 may be made of a flexible and/or elastic material: this material may be one of silicone, silicone based material, rubber and latex.

The duckbill component 77 of the duckbill valve 75 may have a tapered shape, with the tapered portion facing the outlet connector 75b. The tapered shape duckbill component 77 may extend between a passage wide section 77a and a passage closing section 77b for the fluid, so that the infusion fluid flows sequentially from the inlet connector 75a, into the passage wide section 77a, and then through the passage closing section 77b of the duckbill component 77, and afterwards through the outlet connector 75b of the duckbill valve 75.

The passage wide section 77a is connected, in a fluid tight manner, to an exit of the inlet connector 75a inside the inner volume 76a. Thus, the passage wide section 77a is interposed between the passage closing section 77b and the inlet connector 75a of the duckbill valve 75.

The passage closing section 77b is arranged downstream of the passage wide section 77a with respect to the infusion direction ID. Notably, the passage closing section 77b opens into the inner volume 76a of the housing 76. In particular the passage closing section 77b may be not connected to the outlet connector 75b: the passage closing section 77b may indeed be cantilevered within the inner volume 76a of the housing 76 of the duckbill valve 75.

The passage closing section 77b may comprise a deformable slit deformable between an open condition and a closed condition. In the open condition, the passage closing section 77b of the duckbill component 77 allows the infusion fluid to flow from the inlet connector 75a towards the outlet connector 75b: in particular the open condition of the slit defines the open position of the one-way valve as previously described.

In the closed condition, the passage closing section 77b of the duckbill component 77 is closed, thereby preventing fluid to flow through the duckbill component 77. In particular the closed condition of the slit defines the closed position of the one-way valve as previously described.

The passage closing section 77b is configured to move from the closed condition to the open condition when a pressure upstream the duckbill component 77 is greater than a pressure downstream the duckbill component 77. In particular the duckbill component 77 may open when the pressure upstream the duckbill component 77 is at least 3% or at least 5% greater than the pressure downstream the duckbill component 77. In other terms, the duckbill component 77 may open when the upstream pressure is greater than the downstream pressure in the infusion line by a differential pressure comprised between 0,1 Bar and 1 Bar.

The passage closing section 77b, and in particular the slit, may be made of a flexible and/or elastic material, such as one of silicone, silicone based material, rubber and latex. The duckbill component 77 may be made a single piece.

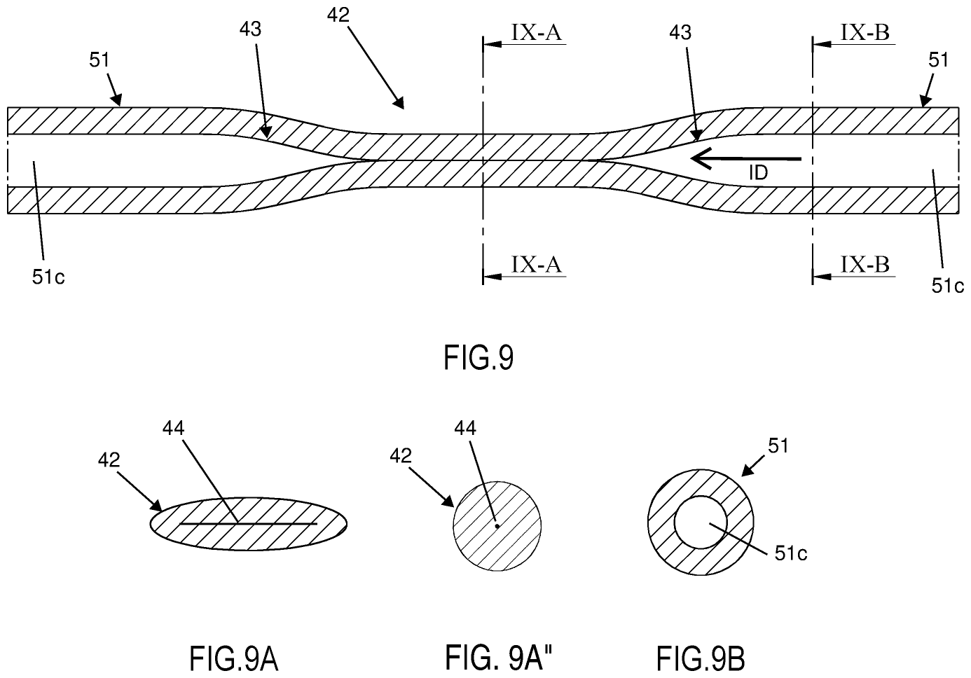

An alternative embodiment of the pressure damper 40 is shown in FIGS. 9A-14B, wherein the pressure damper 40 comprises, instead of the one-way valve, a deformable flow passage restrictor 42 made of an elastic material, such as one of silicone, silicone based material, rubber, PVC, and latex.

The deformable flow passage restrictor 42 is configurable in a rest condition, wherein the deformable flow passage restrictor 42 has a damper lumen 44, which is substantially closed to prevent fluid flow: the deformable flow passage restrictor 42 in the rest condition is shown in FIGS. 9, 11 and 13, and in the respective section views 9A, 9A″; 11A, 11A″; and 13A. Thus, in the rest condition, the deformable flow passage restrictor 42 defines a closure to the fluid passage, so that fluid downstream the pressure damper is fluidly separated from the fluid upstream the pressure damper. In particular, in the rest condition, the damper lumen 44 of the deformable flow passage restrictor 42 is substantially closed in a fluid tight manner.

Alternatively, the damper lumen 44 of the deformable flow passage restrictor 42 may define a damper passage section for the infusion fluid having a size lower than 1 mm², in particular lower than 0.5 mm², more in particular lower than 0.1 mm², more in particular lower than 0.01 mm². In this alternative embodiment, the damper lumen 44 of the deformable flow passage restrictor 42 defines a restriction, instead of the fluid tight closure previously described, for the fluid passage, thereby allowing the fluid to pass through.

Notably, the rest condition is defined when a pressure upstream the deformable flow passage restrictor 42 is substantially equal to a pressure downstream the deformable flow passage restrictor 42. Furthermore, the rest condition is defined when a pressure inside the deformable flow passage restrictor 42 is substantially equal to the atmospheric pressure. This means that the deformable flow passage restrictor 42, when for example disconnected from the infusion line, is configured to keep the rest condition, namely wherein the damper lumen 44 is closed or heavily reduced. In other terms, the shape of the deformable flow passage restrictor 42 when not subjected to external pressure/loads, namely in rest conditions, has the damper lumen 44 closed or heavily reduced: the rest condition is kept by the elastic geometry of the deformable flow passage restrictor 42.

In particular, the deformable flow passage restrictor 42 is configured to return from the infusion condition to the rest condition because of its elastic properties.

FIGS. 9A and 9A″ show, in section view and for exemplary purposes only, two different shapes for the damper lumen 44 of the deformable flow passage restrictor 42 in the rest condition. The damper lumen 44 of the flow passage restrictor 42 of FIG. 9A has a straight line shape: this may originally derive from a circular lumen, which has been collapsed, during fabrication, to close the fluid passage. This leads to a deformable flow passage restrictor 42 having an external elliptical shape, as represented in FIG. 9A.

According to another embodiment shown in FIG. 9A″, the damper lumen 44 in the rest condition may have a dot shape: in this case the deformable flow passage restrictor 42 may have an external circular shape, as represented in FIG. 9A.

Alternatively, in embodiments not shown in the attached drawings, the damper lumen 44 of the deformable flow passage restrictor 42 may be a combination of FIGS. 9A and 9A. For example, a deformable flow passage restrictor 42 may have externally an elliptical shape and a damper lumen 44 having a dot shape when in the rest condition: analogously, a deformable flow passage restrictor 42 may have externally a circular shape and a damper lumen 44 having a line shape, i.e. a straight or curved line shape.

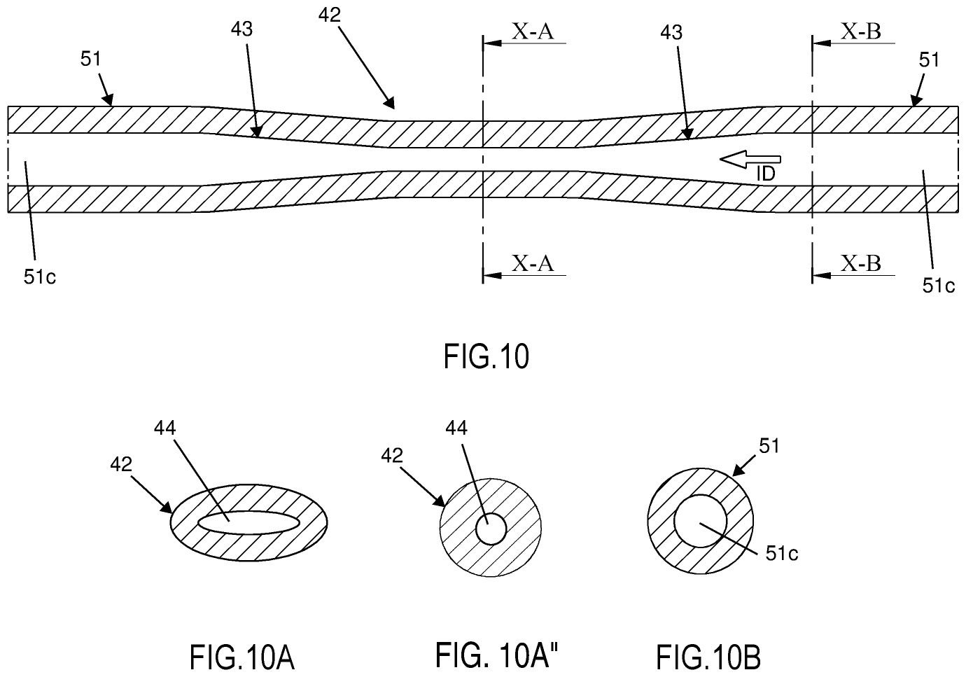

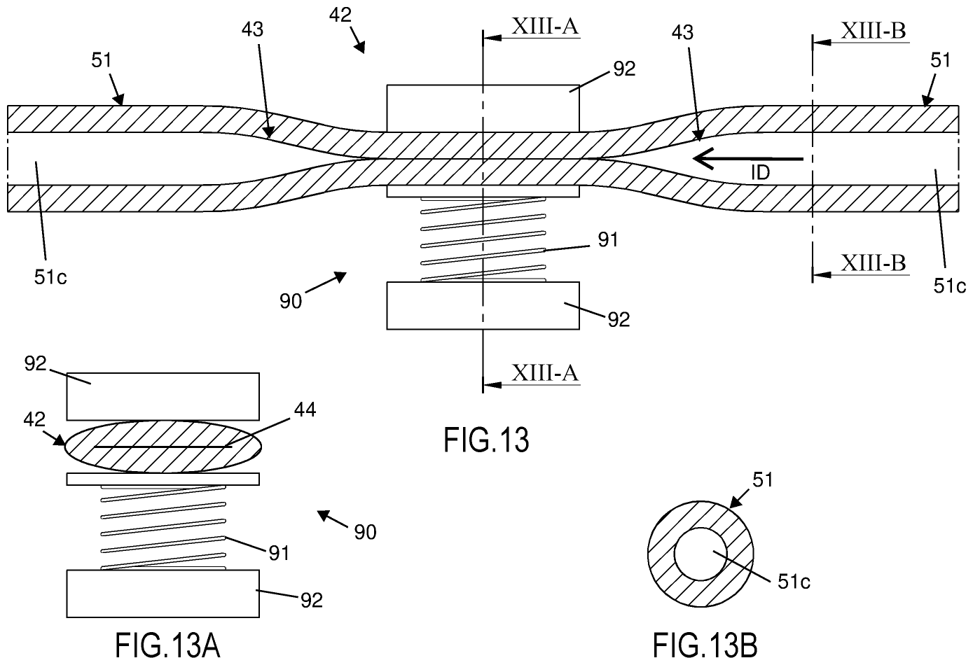

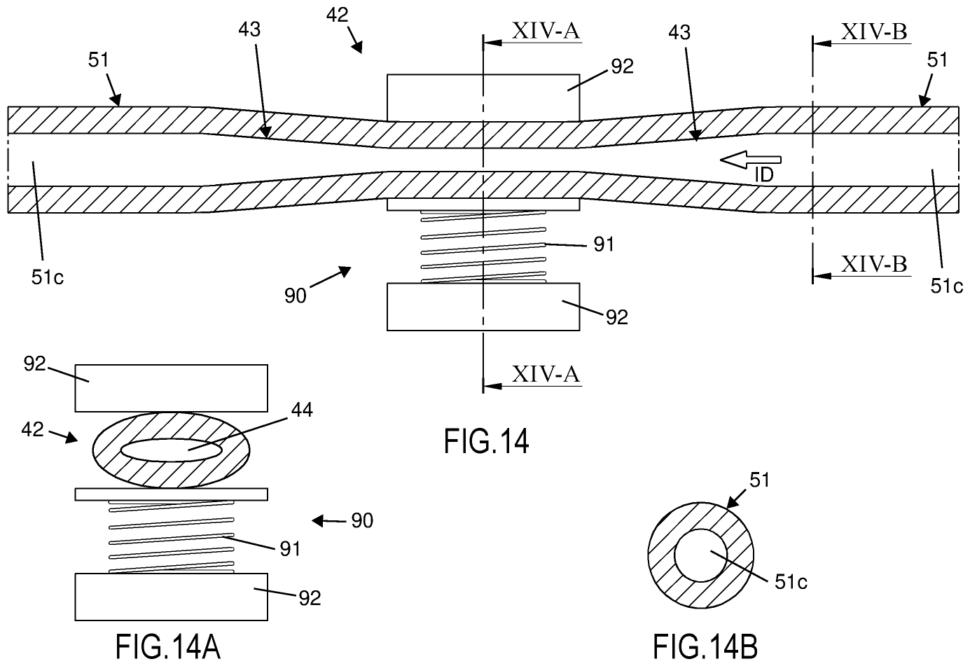

In an embodiment, the infusion line 51 may have an external circular shape, as shown in FIGS. 9B, 10B; 11B, 12B; 13B and 14B, upstream and/or downstream the pressure damper. The external circular shape of the infusion line 51 may be combined with the external elliptical shape of the deformable flow passage restrictor 42, as the external elliptical shape shown in FIGS. 9A, 10A: alternatively, the external circular shape of the infusion line 51 may be combined with the external circular shape of the deformable flow passage restrictor 42, as the external circular shape shown in FIGS. 9A″, 10A″; 11A, 11A″, 12A, 12A″. The external circular shape of the infusion line 51 may be also combined with the embodiment, described afterwards, represented in FIGS. 13 and 14 comprising a squeezing device 90.

The deformable flow passage restrictor 42 is also configurable in an infusion condition, which is shown in FIGS. 10, 12 and 14, and in the respective section views 10A, 10A″; 12A, 12A″; and 14A. When a lumen pressure inside the deformable flow passage restrictor 42 is above a predefined threshold, the damper lumen 44 of the deformable flow passage restrictor 42 switches to the infusion condition, opening the damper lumen 44 to allow passage of the infusion fluid.

The predefined threshold to open the damper lumen 44 may be set greater than 1,02 times the atmospheric pressure, in particular greater than 1,05 times the atmospheric pressure, in particular greater than 1,1 or 1,2 times the atmospheric pressure. Alternatively, the predefined threshold to open the damper lumen 44 may be set greater than 0,05 Bar or 0,1 Bar, optionally greater than 0,2 Bar or 0,3 Bar.

Thus, the elastic feature of the deformable flow passage restrictor 42 allows the damper lumen 44 for extending when subjected to an internal pressure, thereby allowing the infusion condition, and then for retracting elastically in the rest condition when the internal pressure falls below the predefined threshold.

In other terms, the deformable flow passage restrictor 42 switches to the infusion condition, thereby opening the internal damper lumen 44, when the opening forces generated by the pressure inside the deformable flow passage restrictor 42 overcome the closing forces provided by both the structural elasticity of the deformable flow passage restrictor 42 and by the external atmospheric pressure.

If a negative pressure, namely a pressure lower than the atmospheric pressure, is generated by the blood pump in the blood circuit and, therefore, also in the infusion line downstream the pressure damper, said negative pressure also contributes to keep the deformable flow passage restrictor 42 in the rest condition: in this case the differential pressure acting on the pressure damper generates a closing force on the deformable flow passage restrictor 42, thereby providing a further force contribution to keep the damper lumen 44 closed.

The damper lumen 44 of the deformable flow passage restrictor 42 may extend in a flow direction by a length comprised between 5 mm and 100 mm, in particular between 10 mm and 50 mm, optionally between 10 mm and 30 mm.

If the deformable flow passage restrictor 42 has, in the rest condition, a line shaped collapsed damper lumen, as in FIGS. 9A, 11A, and 13A, then in the infusion condition the deformable flow passage restrictor 42 may have a lumen section having a substantially elliptical shape as shown in FIGS. 10A, 12A and 14A.

Alternatively, if the deformable flow passage restrictor 42 has in the rest condition a dot shaped collapsed damper lumen, as in FIGS. 9A″ and 11A″, then in the infusion condition the deformable flow passage restrictor 42 may have a lumen section having a substantially circular shape, as shown in FIGS. 10A″ and 12A″.

As shown in the FIGS. from 9A to 14, the deformable flow passage restrictor 42 may be a tube tract of the infusion line 51. Notably, the deformable flow passage restrictor 42 may made as a single piece. In other terms, the deformable flow passage restrictor 42 may not comprise a plurality of parts assembled together, while it may be fabricated as a single piece, namely a tube tract having the above described features.

Furthermore, the deformable flow passage restrictor 42 may be in one piece with the infusion line: in this case the deformable flow passage restrictor 42 and the infusion line are de facto a seamless single piece.

Alternatively, the deformable flow passage restrictor 42, i.e. a single piece deformable flow passage restrictor 42, may be originally separated from the infusion line: in this case the deformable flow passage restrictor 42 may comprise a first connector configured to be connected to an upstream tract of the infusion line and a second connector configured to be connected to a downstream tract of the infusion line.

A tube wall of the deformable flow passage restrictor 42 may have a thickness substantially equal to a thickness of a tube wall of the rest of the infusion line, in particular substantially equal to a thickness of an upstream tube wall of the infusion line arranged upstream of the deformable flow passage restrictor 42, as shown in FIGS. 9, 10, 13 and 14.

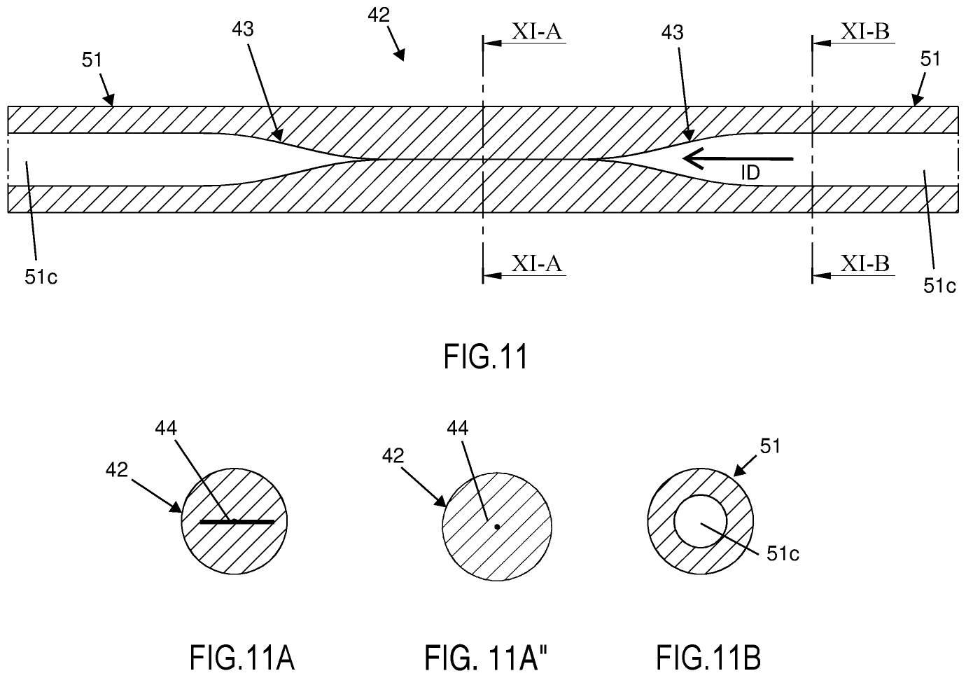

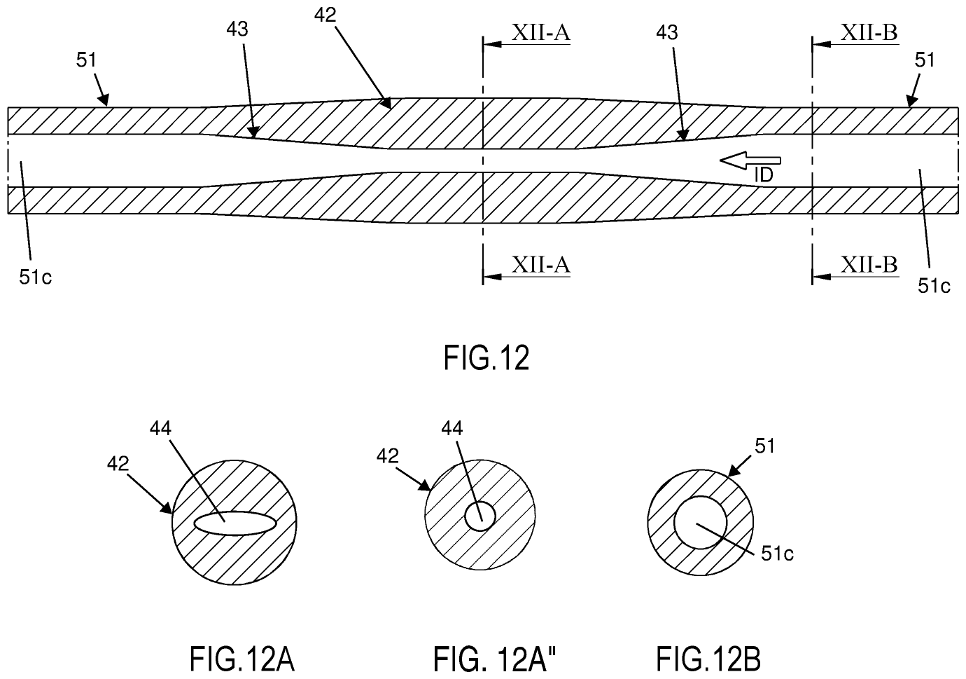

Alternatively, a tube wall of the deformable flow passage restrictor 42 may have a thickness greater than a thickness of a tube wall of the rest of the infusion line, in particular greater than a thickness of an upstream tube wall of the infusion line positioned just upstream the deformable flow passage restrictor 42, as shown in FIGS. 11 and 12.

Beyond the thickness of the tube wall of the deformable flow passage restrictor 42, the deformable flow passage restrictor 42 may be made of the same material of the rest of the infusion line, such as silicone, a silicone-based material, latex, PVC, or rubber.

Furthermore, the deformable flow passage restrictor 42 and the infusion line 51 may be made as a seamless single piece.

The deformable flow passage restrictor 42 may comprise a narrowing lumen section 43, preferably at least arranged upstream the damper lumen 44 and optionally also downstream the damper lumen 44. The narrowing lumen section 43 defines a reduction of the inner lumen of the infusion line before the damper lumen 44. In particular, at least in the rest condition, a lumen of a tube tract of the infusion line 51 upstream the deformable flow passage restrictor 42 may reduce gradually in size towards the damper lumen 44 of the deformable flow passage restrictor 42 defining the narrowing lumen section 43. The tube lumen reduces its size gradually in the narrowing lumen section 43, not instantaneously, in order to allow the inner pressure to open the damper lumen 44 when fluid infusion is needed. The infusion fluid may be pressurized by the infusion pump 54: the pressurized infusion fluid determine an opening force on an internal surface of the narrowing lumen section 43, thereby contributing to open the damper lumen 44, thereby promoting switch from the rest condition to the infusion condition.

The narrowing lumen section 43 may have a conic or truncated cone shape, with straight lateral walls. The narrowing lumen section 43 may define an angle, between the section of the upstream tube tract and the damper section of the deformable flow passage restrictor 42, comprised between 10° and 45°.

Alternatively, the narrowing lumen section 43 may extend in length along a curvilinear line path.

The narrowing lumen section 43 may extend in length along the flow direction by a length of at least 2 mm, in particular at least 5 mm, optionally between 2 mm and 20 mm.

The pressure damper 40 may comprise a squeezing device 90, as shown in FIGS. 13 and 14, operating in thrust on the deformable flow passage restrictor 42, in particular on the tube tract of the deformable flow passage restrictor 42. Notably, the squeezing device 90 is configured to allow the deformable flow passage restrictor 42 to move between the rest condition, namely when the damper lumen 44 is substantially closed or heavily reduced, and the infusion condition, when the damper lumen 44 is opened to allow the infusion fluid to pass through. The deformable flow passage restrictor 42 coupleable to the squeezing device 90 is according to the embodiment previously described and shown in FIGS. 9-14: in particular the deformable flow passage restrictor 42 is elastically deformable to switch between the rest condition and the infusion condition.

The squeezing device 90 is configured to provide a closing force contribution to keep the damper lumen 44 substantially closed in the rest condition, as shown in FIG. 13. Furthermore, the squeezing device 90 is also configured to allow the deformable flow passage restrictor 42 to switch in the infusion condition when a pressure in the infusion line overcomes the predefine threshold. The predefine threshold has been already detailed in description: in particular the predefined threshold to open the damper lumen 44 may greater than 1,02 times the atmospheric pressure, in particular greater than 1,05 times the atmospheric pressure, in particular greater than 1,1 or 1,2 times the atmospheric pressure. Alternatively, the predefined threshold may be greater than 0,05 Bar or 0,1 Bar, optionally greater than 0,2 Bar or 0,3 Bar.

The squeezing device 90 is arranged around the deformable flow passage restrictor 42 and acts in compression on an external surface of the deformable flow passage restrictor 42. For example, the squeezing device 90 may comprise a respective plate/plates 92 acting in thrust on the external surface of the deformable flow passage restrictor 42: the plate 92 may have a flat shape. The plate 92 may extend, along the fluid direction, by a length greater than 1 cm, in particular greater than 2 cm. In particular the plate 92 may extend in length between 1 cm and 5 cm or between 1 cm and 3 cm.

The squeezing device 90 may comprise an elastic element 91, optionally a spiral spring or a flat spring or a spring-like element, configured to keep the rest condition. The elastic element 91 acts in thrust on the external surface of the deformable flow passage restrictor 42, pressing the deformable flow passage restrictor 42, in particular pressing the tube tract of the deformable flow passage restrictor 42. The plate 92 may be interposed in contact between the elastic element 91 and the deformable flow passage restrictor 42.

The elastic element 91 of the squeezing device 90 may be preloaded in the rest condition, so that in the rest condition the squeezing device 90 provides a closing force on the deformable flow passage restrictor 42, to keep the rest condition. Optionally, the preload magnitude of the elastic element 91 may be adjustable, i.e. manually by an operator.

### Extracorporeal Blood Treatment Apparatus 1

With reference to FIG. 1, the numeral 1 globally refers to the extracorporeal blood treatment apparatus, in particular for intensive care therapies, configured to receive the disposable set previously described. The extracorporeal blood treatment apparatus 1 is designed for performing any one of treatments like e.g., hemodialysis, hemofiltration, hemodiafiltration, and ultrafiltration.

The apparatus according to FIG. 1 is particularly designed for continuous renal replacement therapies (CRRT). CRRT systems are configured for delivering very specific treatments designed for patients versing in acute states of illness and who have temporarily lost their kidney function in its entirety. In this respect, CRRT systems may be structurally and/or operationally different from extracorporeal blood treatment systems designed for chronic patient care. In contrast to chronic patients, acute patients temporarily experience complete loss of their kidney function typically due to a contemporaneous state of severe injury or during recovery from surgery. Consequently, acute patients are often extremely weak and typically not in a condition to be submitted to regular dialysis treatment, which may further deteriorate their state and lead to serious and possibly life-threatening complications. Under circumstances as described, CRRT systems are designed to individually treat a patient exhibiting very poor health, without inducing further stress to the patient body, in particular without allowing vital parameters pertaining to the patient's blood to deviate from ideal or near-ideal values. Within the scope of this document CRRT systems are, thus, inherently characterized by one or more of the following features. CRRT involves renal replacement therapy, meaning an adjuvant therapy aimed firstly at facilitating continuous fluid removal in diuretic-resistant or acute renal failure patients. Therefore, CRRT systems inherently require a continuous net fluid removal from the patient. In other words, a CRRT system requires a fluid balance control system, such as a weight loss control system, configured to generate a continuous net weight loss rate (as opposed to merely controlling parameters to enable achieving a desired target weight loss as typically found in chronic patient care). Furthermore, acute patients experience extravascular fluid overload, which cannot be safely removed within a short period of time (e.g. within a few hours of chronic treatment) without causing potentially severe consequences (e.g. hypovolemic shock, arrhythmia, hypoxemia, hypoventilation, etc.). Therefore, a CRRT system must inherently include a much more accurate control over system parameters, in particular flow rates, in order to ensure that the required low flow rates of both blood circulating extra-corporeally and of treatment fluid (infused in the extracorporeal circuit or diffused through the dialyzer) are used. Moreover, CRRT treatment is performed continuously (e.g. for days or even weeks, without interruption/with minimal interruptions e.g., downtimes to change bags). Therefore, treatment settings in CRRT are based on flow rate settings, rather than settings pertaining to some specified treatment time (which would be unknown as acute patients may require treatment for an unknown time). Consequently, operation of CRRT systems cannot be based on some pre-defined absolute weight loss to be achieved, but rather on a meticulously controlled fluid balance in the patient, requiring continuous adjustments to a number of operating parameters, which have to be controlled and maintained during the entire (and a priori unknown) treatment time, based on a set weight-loss rate. Additionally, CRRT renal replacement therapy involves therapy substituting kidney functions for a relatively long time period and, thus, a CRRT system further requires at least either fresh dialysis liquid exchange in the dialyzer (in order to remove unwanted substances from blood and to add desired substances to the blood by diffusion) and/or fresh infusion fluid in combination with ultrafiltration (in order to remove unwanted substances from blood and to add desired substances to the blood by convection).

At least for the reasons set forth above, CRRT systems need to exhibit specific technical features enabling the system to:
allow setting of a weight loss rate,
continuously remove excess water in accordance with a set weight loss rate,
operate continuously at comparably low flow rates compatible with CRRT, and
Balance ion equilibrium by means of proper dialysis being performed and/or by means of substitution fluid continuously being delivered at controlled flow rates.

Finally, in order to set up a CRRT apparatus as soon as possible, the CRRT machine is dressed using an integrated disposable set 100, wherein all the lines and the filtration unit are grouped together and already properly connected in the disposable set. Further, all the fluids are contained in pre-packaged bags (dialysis fluid or replacement fluids in bags of e.g., 2, 5 or 10 litres each) or pre-packaged syringes (heparin and/or concentrated calcium replacement solution).

The apparatus 1 of FIG. 1 has an extracorporeal blood circuit 17, which takes blood from a patient P, for instance through a needle or a catheter or an implanted port or other access device (not shown), introduced into a vein or artery of said patient, and through the blood withdrawal line 6 takes said blood, for instance continuously, to the filtration unit 2.

The blood passes through the primary chamber 3 of the filtration unit 2 and, through the blood return line 7, the treated blood is carried back to the patient. In the example of FIG. 1, the connection with the infusion line 51 is provided immediately downstream from the blood collecting zone on the blood withdrawal line 6. In particular, the machine is equipped with the infusion substance source 10, containing an infusion fluid comprising for example one between bicarbonate, citrate or citric acid a replacement fluid, saline, and a regional anticoagulant solution; by using the infusion pump 54, for instance a peristaltic pump, it is possible to control the fluid flow within said infusion line 51 by introducing the infusion fluid directly into the blood with a direct connection to the blood withdrawal line 6. After defining a direction of fluid (blood) circulation 200 (during normal use of the apparatus) from the blood withdrawal line 6 towards the filtration unit 2 and from the latter through the blood return line 7 towards the patient P, a known blood pressure sensor 48, which shall not be described in further detail, may be arranged immediately downstream the infusion line 51, namely between the infusion line 51 and the blood pump 21. The apparatus comprises the blood pump 21 for controlling and managing the suitable blood flow Q_{b} in the circuit. The blood pump 21 is generally a peristaltic pump acting either on the blood withdrawal line (as shown e.g. in FIG. 1) and/or on the blood return line. An operator may enter a set value for the blood flow rate Q_{b} through a user interface 15 and a control unit 12, during treatment, is configured to control the blood pump based on the set blood flow rate. Note that, alternatively, the blood pump 21 may be automatically controlled with no need of user input/prescription: in that case control unit 12 may control the blood pump 21 at a prefixed flow rate or at a flow rate calculated based on other parameters such as, for instance, other flow rates and constraints set by the medical operator (as apparent from the following description). In addition, or alternatively, the blood pump may also be controlled based on pressure; if the blood pump 21 is controlled based on the pressure signal detected upstream the blood pump then a pressure sensor 48 is present in the tract of bloodline upstream the blood pump 21: for instance the control unit 12 may be designed to drive the blood pump in a manner to keep the pressure detected by pressure sensor 48 within a prefixed range, or below a prefixed threshold.

Following the direction of blood circulation 200, in case the apparatus is also configured to remove CO₂ a gas exchanger 46 for removing CO₂ from circulating blood may be connected to the blood circuit. The gas exchanger 46 is in fluid communication with the blood circuit 17 to receive extracorporeal blood, allow CO₂ removal from blood and returning blood to the blood circuit at a downstream point. FIG. 1 shows a gas exchanger 46 placed upstream the filtration unit 2; however, the gas exchanger may be alternatively positioned downstream the filtration unit on the blood return line 7. Notably, in case the CO₂ gas exchanger 46 is positioned downstream the filtration unit 2 on the blood return line 7, degassing in the blood withdrawal line increased its importance because air bubble in the filtration unit may cause blood clotting. As mentioned, blood circulation direction during normal use of the apparatus is indicated in FIG. 1 with an arrow 200 which also represent the blood flow rate Q_{b} direction during treatment. The gas exchanger 46 is connected in series with the filtration unit 2 and is placed downstream the injection point 50 where the infusion solution is delivered to extracorporeal blood. The gas exchanger 46 has a blood chamber and a gas chamber separated by a membrane permeable to gases, in particular CO₂; the gas exchanger comprise a gas inlet, which may be connected to a gas source, such as the medical gas supply system in a hospital to receive pressurized air or oxygen for example, and a gas outlet in fluid communication with the gas chamber to discharge exhausted gas having removed CO₂ from extracorporeal blood. The blood inlet and the blood outlet put the extracorporeal blood circuit 17 in fluid communication with the gas exchanger blood chamber. Clearly, the gas exchanger 46 may not be part of the dialysis apparatus should CO₂ removal be not necessary for the treatment.

Then, another pressure sensor 49 may be provided on the blood withdrawal line 6 for controlling the correct flow within the blood circuit: the pressure sensor 49 is interposed between the blood pump 21 and the filtration unit 2.

After passing through the primary chamber 3 of the filtration unit 2, where the suitable exchanges of substances, molecules and fluids occur by means of a semipermeable membrane, the treated blood enters the blood return line 7, first passing through the air separator 19, commonly known as “bubble trap”, designed so as to ensure the detection and removal of air bubbles present in the blood. The treated blood getting out of the air separator 19, before being returned to the patient P passes through an air bubble sensor 55 verifying the absence of said dangerous formations within the treated blood that has to be re-introduced in the patient's blood circulation. Immediately downstream from the bubble sensor 55, the safety valve 20 (or venous clamp) is placed which, in case of alarm, may block the blood flow towards the patient. In particular, should the bubble sensor 55 detect the presence of air in the blood flow, the machine through safety valve 20 would be able to block immediately the passage of blood so as to avoid any consequence to the patient. A corresponding safety valve 27 (or arterial clamp) is present on the blood withdrawal line close the patient vascular access to fully isolate the patient from the extracorporeal blood circuit in case of need. Downstream from the safety valve 20, the treated blood is then carried back to the patient P undergoing therapy. The extracorporeal blood treatment apparatus of FIG. 1 is equipped with a dialysis fluid circuit 32, which is also provided with at least a dialysis supply line 8 leading into the filtration unit 2 and with an effluent (or dialysate) line 13 from the filtration unit. At least a primary fluid container, defining said dialysis liquid source 14, is designed to supply the supply line 8 of the dialysis fluid circuit 32 (generally the primary fluid container shall consist of one or more bags containing a suitable dialysis liquid). The supply line 8 includes actuator/s for conveying fluid such as at least a dialysis fluid pump 25 (in the embodiment of FIG. 1a peristaltic pump) for controlling the flow rate Q_{dial} of dialysis liquid from the bag and for defining a direction 200 of dialysis fluid circulation. Downstream from the dialysis fluid pump 25 in the direction of circulation 200 there is a branching 56 splitting the dialysis supply line 8 up into an intake branch 57 and an infusion branch 58. In particular, the infusion branch 58 is connected to the blood return line 7 of the blood circuit 17. In other words, through said infusion branch 58 it is possible to obtain a post-infusion directly in the blood line 17 using the content of the primary fluid container. Conversely, the intake branch 57 conveys the fluid directly to the filtration unit 2 and in particular to the secondary chamber of said unit. The dialysis fluid circuit 32 is further equipped with a selector 59 for determining the percentages of fluid flow within the infusion branch 58 and the intake branch 57. Generally said selector 59, usually placed near the branching 56, may be positioned at least between a first operating condition in which it allows the passage of fluid in the intake branch 57 and blocks the passage in the infusion branch 58, and a second operating condition in which it allows the passage of fluid in the infusion branch 58 and blocks the passage in the intake branch 57. In other words, said selector 59 may consist of a valve element operating on the dialysis fluid circuit 32 by alternatively blocking the passage of fluid in either branch. Suitable selectors may be alternatively provided, which are able to establish a priori the amount of liquid that has to pass through both branches simultaneously. It will also be possible to vary the percentages of fluid in either branch as a function of time and of the pre-established therapies. The dialysis liquid through the intake branch 57 gets into the secondary chamber 4 of the filtration unit 2. In particular, the primary chamber 3 through which the blood flow passes is separated from the secondary chamber 4 through which the dialysis liquid passes through the semipermeable membrane 5 ensuring the suitable passage of the dangerous substances/molecules and of fluid from the blood towards the dialysis liquid mainly through convection and diffusion processes, and also ensuring through the same principles the passage of substances/molecules from the dialysis liquid towards the blood. The dialysis fluid then gets into the effluent line 13 and passes through a suitable effluent pressure sensor 60. An actuator is provided for conveying fluid, for instance a dialysate pump 26 controlling the flow rate Q_{eff} in the effluent line 13 within the fluid circuit 32. Also said pump will generally be a peristaltic pump. The fluid to be eliminated then passes through a blood detector 61 and is conveyed into a collection container or bag 62.

A further infusion line 51 for feeding fluid into the blood return line 7 of the blood circuit 17 may be provided. In particular, the infusion fluid is taken from at least an auxiliary container 64 and is sent directly to the blood return line 7 of the blood circuit 17 through actuator/s for conveying fluid, generally an infusion pump 65 (in the example a peristaltic pump) controlling its flow rate Qᵣₑₚ−total replacement flow rate. In particular, the infusion liquid may be introduced directly into the air separator 19. As can also be inferred, the infusion branch 58 of the dialysis fluid circuit 32 and the infusion line 63 are equipped with a common end length 66 letting fluid to enter into the blood circuit 17. Said intake end length 66 is placed downstream from the infusion pump 65 with respect to a direction of infusion and carries the fluid directly into the air separator 19. Further, referring to the diagram in FIG. 1, the infusion line 63 comprises at least a pre-infusion branch 67 connected to the blood withdrawal line 6 of the blood circuit 17. In further detail, downstream from the infusion pump 65 with respect to the direction of infusion, there is an infusion branching 68 splitting the infusion line 63 up into the pre-infusion branch 67 and post-infusion branch 69. The pre-infusion branch 67, in particular, carries the fluid taken from the bag 64 into the blood withdrawal line 6 of the blood circuit 17 downstream from the blood pump 21 and downstream the gas exchanger 46 with respect to the direction of blood circulation. Conversely, the post-infusion branch 69 is connected directly to the common end length 66. The infusion line 63 further comprises a selector 70 for determining the percentage of liquid flow to be sent to the post-infusion branch 69 and to the pre-infusion branch 67. The selector 70 placed near the branching 68 may be switched between at least a first operating condition in which it allows the passage of fluid in the pre-infusion branch 67 and blocks the passage in the post-infusion branch 69, and at least a second operating condition in which it allows the passage of fluid in the post-infusion branch 69 and blocks the passage in the pre-infusion branch 67. Obviously, as in the case of the selector 59 present on the dialysis fluid circuit 32, also the other selector 70 will be able to determine the percentage of fluid that has to pass in each of the two branches and to possibly vary it in time in accordance with the planned therapies. Moreover, the selector 59 and the other selector 70 will generally, though not necessarily, be of the same nature. Notably, the flow rate through the pre-infusion branch/line 67 may be determined by proper control of the infusion pump 65 and other selector 70; the control unit 12 may receive a pre-infusion ratio of replacement fluid flow PRE (a value between 0 and 1) and determine the pre-infusion flow rate Q_{rep·pre} based on the pre-infusion ratio PRE and on total replacement flow rate Qᵣₑₚ. In particular, Q_{rep·pre}=PRE·Qᵣₑₚ. Alternatively, post-infusion ratio may be used symmetrically, or a ratio between pre- and pos-infusion rate may be used as well (R=Q_{re·pre}/Q_{rep·post}).

The apparatus may be equipped with scales 71 for determining at least the weight of the primary fluid container 14 and/or of the auxiliary fluid container 64 and/or of the infusion substance source 10 and/or of the collection container 62. In particular, said scales 71 comprises weight sensors, for instance respective scales A, B, C, D and E (for example at least an independent sensor for each fluid bag associated to the machine). In particular, there will be at least four of said scales, each being independent from the other, and each one measuring the respective weight of a bag. It should then be pointed out that there is a control unit or CPU 12 active (at least) on the blood circuit 17 and in particular active on the pressure sensor 48 for reading pressure values, on the blood pump 21, on the gas exchanger 46, on the other pressure sensor 49, and on the device for detecting the presence of air bubbles 55 and on the respective safety valves 20, 27. The control unit 12 has also to control the dialysis fluid circuit 32 and, in particular, shall be input with the data detected by the scales A, B, C, D and (possibly) E and, concerning the weight of the bag 14, and shall act on the pump 25, on the selector 59, on the pressure sensor 60, then on the dialysate pump 26 and shall eventually receive the data detected by the scale A whose function is to determine the weight of the collection container 62. The control unit 12 shall also act on the infusion line 63 checking the weight of the auxiliary container 64 (checked by the scale C) and will be able to control both the infusion pump 54 65 and the other selector 70. The control unit 12 shall also act on the infusion line 51 detecting the weight of the infusion substance source 10 through the scale B and suitably controlling the infusion pump 54 according to the treatments to be carried out as below detailed and explained. However, the apparatus of FIG. 1 may be alternatively (or additionally) provided with a systemic anticoagulation system, such as a syringe pump 9 for injecting heparin downstream the blood pump 21.

The control unit 12 is also connected to a memory and to user interface, for instance a graphic user interface, which receives operator's inputs and displays the apparatus outputs. For instance, the graphic user interface may include a touch screen, a display screen and/or hard keys for entering user's inputs or a combination thereof.

The control unit 12 is also connected to the blood pump 21 and configured to control the blood pump 21 to determine a blood flow rate in the blood withdrawal line 6. During an operating condition of the extracorporeal blood treatment, the control unit is configured to define a treatment condition wherein the blood pump 21 is set at a flow rate comprised between 50 ml/min and 600 ml/min, in particular between 100 ml/min and 350 ml/min, more in particular between 200 ml/min and 300 ml/min. Of course, the blood flow rate is set by the physician and may vary depending on various factors including the vascular access, the patient conditions, and the type of treatment.

In any case, during the patient treatment, the access negative pressure is experienced in the blood withdrawal line 6 upstream the blood pump tract 6p engaged by the blood pump 21.

On the other hand, the infusion pump 54 is operatively connected to the control unit 12 which is configured to selectively control the infusion pump 54 to promote the infusion fluid to flow within the infusion line 51 and to deliver the infusion fluid into the blood withdrawal line 6: the control unit 12 may be configured to control the infusion pump 54 to set the flow rate of the infusion fluid typically between 200 ml/h and 4000 ml/h, in particular between 500 ml/h and 2000 ml/h, more in particular at a flow rate substantially close to 1000 ml/h and lower than 1600 ml/h. Also in this regard, the type of treatment, the infusion bag content, and other conditions determine the set infusion rate, which is however normally set in the above discussed ranges.

The control unit may also be configured to deactivate the infusion pump 54 to arrest delivery of the infusion fluid.

According to an embodiment wherein the disposable set comprises the one-way valve 46, the control unit may be configured to define a first condition and a second condition.

In the first condition, the infusion pump 54 is active to generate the infusion fluid flow towards the blood withdrawal line 6 and the blood pump 21 is active to determine the blood flow in the blood circuit of the disposable set. In the first condition, the differential pressure across the one-way valve is higher than the preset opening pressure threshold of the one-way valve 46. This pressure difference causes the one-way valve 46 to switch to or to maintain the open position, allowing thereby the infusion fluid to be delivered into the blood withdrawal line 6. In other terms, the infusion pump 54 generates an over pressure which, combined with the low pressure caused by the blood pressure, determines the aperture of the one-way valve 46.

In the second condition, the infusion pump 54 is stopped to prevent infusion of the infusion fluid into the blood withdrawal line 6, while the blood pump is active: in the second condition the one-way valve 46 closes and prevents the infusion line 51 to be in fluid communication with the blood line. The access negative pressure is prevented to extend in the infusion line 51, thereby avoiding degassing of the infusion fluid.

The infusion pump 54 may be configured to generate a head pressure greater than a predefined threshold to open either the one-way valve 46; 75 or the deformable flow passage restrictor 42. In particular the infusion pump 54 may be configured to generate, when an infusion of the infusion fluid is requested in the blood circuit 17, an head pressure greater than 0,05 Bar or 0,1 Bar, optionally greater than 0,2 Bar or 0,3 Bar. Notably, this head pressure is expressed as a relative pressure with respect to the atmospheric pressure: thus an head pressure of 0,05 Bar expressed as a relative pressure substantially corresponds to an absolute pressure of 1,05 Bar.

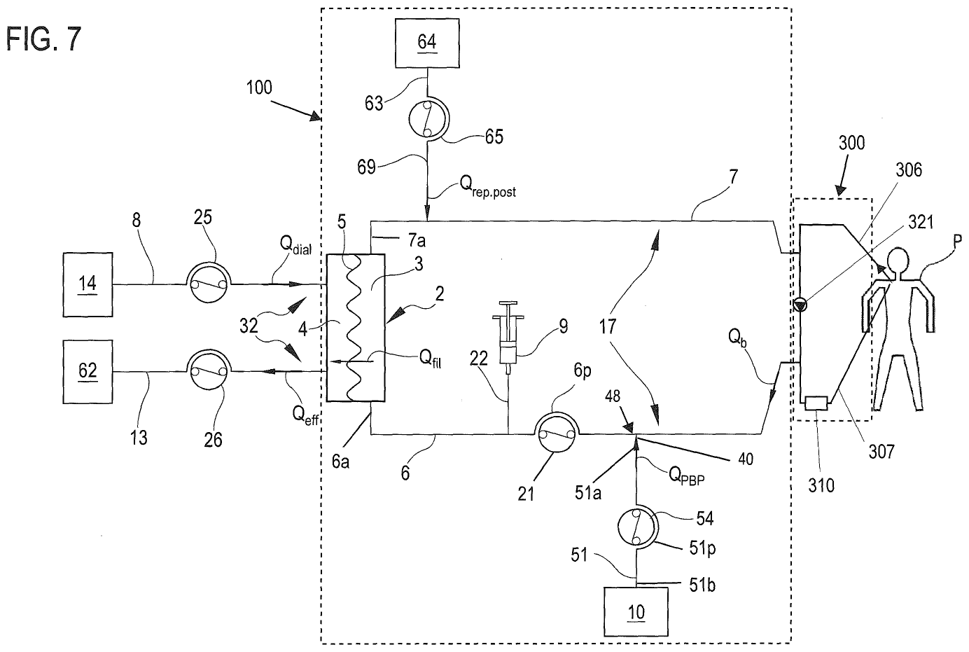

The infusion pump 54 may comprise an occlusive pump or a volumetric pump, for example a peristaltic pump. FIG. 7 shows an embodiment wherein a disposable set 100 for extracorporeal blood treatment, for example a dialysis treatment, is connected to an Extra Corporeal Membrane Oxygenation ECMO circuit 300, which is in turn configured for connection to the patient. In other terms, the ECMO circuit 300 is fluidly interposed between the disposable set 100 and the patient's vascular accesses.

The ECMO circuit is only very schematically represented in order to show a further extracorporeal blood flow circulation. Clearly, the ECMO circuit includes all necessary lines and components for proper working. No further details are provided about a detailed embodiment of the ECMO circuit: anyhow, the skilled person knows the key features of an ECMO circuit and the main elements/devices associated without further explanation. Notably, the ECMO circuit is not per se part of the present invention, while it is an extra circuit where the disposable set 100 of the present invention may be coupled to, instead of directly connecting the disposable set 100 to the patient. Indeed, when blood oxygenation and a dialysis treatment are proper, a layout as the one proposed in FIG. 7 may be implemented in order to avoid multiple blood accesses to the patient.

Clearly any suitable connection of the extracorporeal blood circuit 100 to the ECMO apparatus may be used. The blood withdrawal line 6 and/or the blood return line 7 may be connected to respective blood lines of the ECMO apparatus wherein positive or negative pressure occurs. Two examples of possible connection are below briefly described. However, what is relevant here is that: (i) the extracorporeal blood circuit 100 may not be directly connected to the patient access (ii) any one of the blood withdrawal line 6 and the blood return line 7 may experience a negative pressure regimen and therefore the corresponding pressure damper may be usefully applied at the junction point of any infusion line injecting into the blood circuit where there is a negative pressure in the blood at the junction point.

According to the embodiment of FIG. 7, the blood withdrawal line 6 of the disposable set 100 is connected to the ECMO circuit 300 downstream the blood pump 321 of the ECMO circuit, while the blood return line 7 of the disposable set 100 is connected to the ECMO circuit 300 upstream the blood pump 321 of the ECMO circuit. In this case, the blood withdrawal line 6 of the disposable line experience a positive pressure: thus, the pressure at the cross between the blood withdrawal line 6 and the infusion line 51 in the disposable set 100 prevents degassing of the infusion fluid. However, the blood return line 7 may experience a negative pressure regimen. In this case, the use of a pressure damper arranged proximate or at the first end of the post infusion line 69 may be used to prevent degassing in the post infusion line itself. The pressure damper is configured to prevent, or reduce an amount of, the negative pressure to extend in the post infusion line 69 upstream the pressure damper. The same embodiments of pressure dampers already discussed in the previous sections may be used here.

In a second ECMO embodiment not shown in the attached figured, the blood withdrawal line 6 of the disposable set 100 may be connected to the ECMO circuit 300 upstream the blood pump 321 of the ECMO circuit, while the blood return line 7 of the disposable set 100 may be connected to the ECMO circuit 300 downstream the blood pump 321 of the ECMO circuit. In this case, the pressure at the fluid access 48 between the blood withdrawal line 6 and the infusion line 51 in the disposable set 100 may be negative. Thus, the infusion fluid in the infusion line 51 may be subjected to a low pressure, which may cause fluid degassing. Thus, a disposable set according to the present invention may allow avoiding fluid degassing in the fluid line 51 when connected to an ECMO circuit, when the blood line having the infusion line 51 is connected to the ECMO circuit upstream the blood pump 321 of the ECMO circuit.

1-38. (canceled)

39. Disposable set for an extracorporeal blood treatment apparatus, the disposable set comprising:
a filtration unit;
a blood circuit comprising:
a blood withdrawal line extending between a first end connected to the filtration unit and a second end,
a blood return line extending between a first end connected to the filtration unit and a second end;
the blood withdrawal line comprising a blood pump tract configured to be engaged by a blood pump of the extracorporeal blood treatment apparatus which is configured to determine a blood flow, wherein, at least during an operating condition, an access negative pressure is experienced upstream said blood pump tract, said blood flow in the blood circuit being in a direction from the blood withdrawal line towards the filtration unit and from the filtration unit through the blood return line;
an infusion line extending between a first end connected to the blood withdrawal line at a fluid access upstream the blood pump tract, and a second end configured to connect to an infusion substance source, wherein the blood pump tract is interposed between the filtration unit and the first end of the infusion line;
wherein the disposable set further comprises a pressure damper arranged proximate or at the first end of the infusion line, the pressure damper configured to prevent or reduce passage of the access negative pressure to the infusion line upstream the pressure damper.

40. Disposable set according to claim 39, wherein the pressure damper comprises a one-way valve configured to allow fluid passage only in an infusion direction directed from the infusion line towards the withdrawal line, and is configured to move between an open position, in which the fluid passage is allowed in said infusion direction, and a closed position, in which fluid passage is prevented in both directions, the one-way valve being preset at an opening pressure threshold to switch between the closed position and the open position and vice versa, so that:
when a differential pressure between an upstream section and a downstream section of the one-way valve is equal to or higher than said opening pressure threshold, the one-way valve is configured to switch to or to maintain the open position, and
when said differential pressure is lower than said opening pressure threshold, the one-way valve is configured to switch to or to maintain the closed position.

41. Disposable set according to claim 40, wherein the differential pressure is defined between a high pressure zone upstream the one-way valve, and a low pressure zone downstream the one-way valve according to the infusion direction, said high pressure being higher than said low pressure, and wherein the one-way valve prevents a fluid passage in a direction from the withdrawal line to the infusion line in any condition.

42. Disposable set according to claim 40, wherein the one-way valve comprises an internal diaphragm movable between the open position and the closed position, the internal diaphragm being preloaded in the closed position, said preload defining the preset opening pressure threshold.

43. Disposable set according to claim 42, comprising a Luer lock connector housing said one-way valve, wherein said internal diaphragm is within an internal fluid passage of the Luer lock connector.

44. Disposable set according to claim 40, wherein said one-way valve is a duckbill valve comprising a housing defining an inner volume, said housing comprising:
an inlet connector connected to an upstream tract of the infusion line;
an outlet connector connected to a downstream tract of the infusion line;
a duckbill component arranged in the inner volume of the housing and configured to allow the infusion fluid to flow from said inlet connector to said outlet connector and to prevent fluid to flow backwards from said outlet connector to said inlet connector, wherein the duckbill component is made of a flexible and/or elastic material.

45. Disposable set according to claim 44, wherein said duckbill valve is arranged on the infusion line at a distance from the first end of the infusion line comprised between 0.1 cm and 6 cm.

46. Disposable set according to claim 44, wherein the duckbill component of the duckbill valve comprises a tapered shape extending between a passage wide section and a passage closing section for the fluid,
and wherein the duckbill valve is configured to allow the infusion fluid to flow sequentially from the inlet connector, into the passage wide section, and then through the passage closing section of the duckbill component, and afterwards through the outlet connector of the duckbill valve,
the passage wide section being connected, in a fluid tight manner, to an exit of the inlet connector inside the inner volume.

47. Disposable set according to claim 46, wherein the passage closing section opens into the inner volume of the housing, wherein the passage closing section is not connected to the outlet connector,
and wherein the passage closing section is cantilevered within the inner volume of the housing of the duckbill valve.

48. Disposable set according to claim 46, wherein the passage closing section comprises a deformable slit deformable between:
an open condition, wherein the passage closing section of the duckbill component allows the infusion fluid to flow from the inlet connector towards the outlet connector, said open condition of the slit defining the open position of the one-way valve; and
a closed condition, wherein the passage closing section of the duckbill component is closed preventing fluid to flow through the duckbill component, said closed condition of the slit defining the closed position of the one-way valve.

49. Disposable set according to claim 40, wherein the preset opening pressure threshold of the one-way valve corresponds to a differential pressure value higher than 160 mmHg.

50. Disposable set according to claim 40, wherein the preset opening pressure threshold of the one-way valve corresponds to a differential pressure value comprised between 160 and 500 mmHg.

51. Disposable set according to claim 40, wherein the preset opening pressure threshold of the one-way valve is set within +/−100 mmHg of a maximum negative pressure allowed at the fluid access during standard working condition of the extracorporeal blood treatment apparatus.

52. Disposable set according to claim 40, wherein said one-way valve is arranged on the infusion line at a distance from the first end of the infusion line no longer than 6 cm.

53. Disposable set for an extracorporeal blood treatment apparatus, the disposable set comprising:
a filtration unit;
a blood circuit comprising:
a blood withdrawal line extending between a first end connected to the filtration unit and a second end,
a blood return line extending between a first end connected to the filtration unit and a second end;
the blood withdrawal line including a blood pump tract configured to be engaged by a blood pump of the extracorporeal blood treatment apparatus which is configured to determine a blood flow, wherein, at least during an operating condition, an access negative pressure is experienced upstream said blood pump tract, said blood flow in the blood circuit being in a direction from the blood withdrawal line towards the filtration unit and from the filtration unit through the blood return line;
an infusion line extending between a first end connected to the blood withdrawal line at a fluid access upstream the blood pump tract, and a second end configured to connect to an infusion substance source, wherein the blood pump tract is interposed between the filtration unit and the first end of the infusion line;
wherein the disposable set further comprises a pressure damper arranged proximate or at the first end of the infusion line and comprising a deformable flow passage restrictor made of an elastic material, the pressure damper configured to prevent or reduce passage of the access negative pressure to the infusion line upstream the pressure damper, and
wherein said deformable flow passage restrictor is configurable between:
a rest condition in which the deformable flow passage restrictor has a damper lumen which is closed to prevent fluid flow or which defines a damper passage section for the infusion fluid having a size lower than 1 square millimeter,
an infusion condition in which a lumen pressure inside the deformable flow passage restrictor is above a predefined threshold, the damper lumen of the deformable flow passage restrictor is configured to open to allow transit of the infusion fluid.

54. Disposable set according to claim 53, wherein said predefined threshold to open the damper lumen is greater than 1.02 times atmospheric pressure.

55. Disposable set according to claim 53, wherein the damper lumen of the deformable flow passage restrictor extends in a flow direction by a length comprised between 5 mm and 100 mm, the deformable flow passage restrictor having, in the rest condition, a damper lumen collapsed having a shape in section between a line and a dot.

56. Disposable set according to claim 53, wherein said deformable flow passage restrictor is a tube tract of the infusion line, said tube tract of the deformable flow passage restrictor having a flattened shape, and wherein said tube tract has a section, orthogonal to a fluid direction, having an external elliptic shape.

57. Disposable set according to claim 53, wherein the deformable flow passage restrictor is a tube tract of the infusion line, and wherein:
a tube wall of the deformable flow passage restrictor has a thickness equal to a thickness of an upstream tube wall of the infusion line arranged upstream of the deformable flow passage restrictor, or
a tube wall of the deformable flow passage restrictor has a thickness greater than a thickness of an upstream tube wall of the infusion line positioned just upstream the deformable flow passage restrictor.

58. Disposable set according to claim 53, wherein, at least in the rest condition, a lumen of a tube tract of the infusion line upstream the deformable flow passage restrictor reduces gradually in size towards the damper lumen of the deformable flow passage restrictor defining a narrowing lumen section.

59. Disposable set according to claim 58, wherein the narrowing lumen section extends in length along a flow direction between 2 mm and 20 mm, the narrowing lumen section extending in length along a curvilinear or straight line path.

60. Disposable set according to claim 53, wherein the pressure damper comprises a squeezing device operating in thrust on a tube tract of the deformable flow passage restrictor, the squeezing device being configured to:
provide a closing force contribution to keep the damper lumen closed in the rest condition, and
allow the deformable flow passage restrictor to switch in the infusion condition when a pressure in the infusion line upstream the pressure damper overcomes a predefine threshold.

61. Disposable set according to claim 60, wherein the squeezing device is arranged around the deformable flow passage restrictor and acts in compression on an external surface of the deformable flow passage restrictor.

62. Disposable set according to claim 60, wherein the squeezing device comprises an elastic element acting in thrust on the external surface of the deformable flow passage restrictor, the elastic element of the squeezing device being preloaded in the rest condition.

63. Disposable set according to claim 62, wherein the preload of the elastic element is adjustable.

64. Disposable set according to claim 60, wherein the squeezing device comprises one or more plates acting in thrust on the external surface of the deformable flow passage restrictor along a length between 1 cm and 5 cm.

65. Disposable set for an extracorporeal blood treatment apparatus, the disposable set comprising:
a filtration unit;
a blood circuit comprising:
a blood withdrawal line extending between a first end connected to the filtration unit and a second end,
a blood return line extending between a first end connected to the filtration unit and a second end;
the blood withdrawal line including a blood pump tract configured to be engaged by a blood pump of the extracorporeal blood treatment apparatus which is configured to determine a blood flow, wherein, at least during an operating condition, an access negative pressure is experienced upstream said blood pump tract, said blood flow in the blood circuit being in a direction from the blood withdrawal line towards the filtration unit and from the filtration unit through the blood return line;
an infusion line extending between a first end connected to the blood withdrawal line at a fluid access upstream the blood pump tract, and a second end configured to connect to an infusion substance source, wherein the blood pump tract is interposed between the filtration unit and the first end of the infusion line;
wherein the disposable set further comprises a pressure damper arranged proximate or at the first end of the infusion line, the pressure damper being configured to prevent or reduce passage of the access negative pressure to the infusion line upstream the pressure damper,
wherein the pressure damper is a flow passage restrictor:
the flow passage restrictor having a damper passage section configured to allow the infusion fluid to pass through, and
the infusion line having a respective fluid passage section configured to allow the infusion fluid to pass through,
wherein the damper passage section is at least 50% smaller than the fluid passage section.

66. Disposable set according to claim 65, wherein the damper passage extends by a length comprised between 30 mm and 200 mm, and the damper passage section has a diameter comprised between 0.3 mm and 0.6 mm.

67. Disposable set according to claim 65, wherein either:
the damper passage section has a diameter equal to 0.5 mm and extends by a length equal to 145 mm±15 mm, or
the damper passage section has a diameter equal to 0.4 mm and extends by a length equal to 60 mm±10 mm,
and wherein said diameter is constant over said length.

68. Disposable set according to claim 65, wherein the flow passage restrictor comprises a septum defining the damper passage section axially extending:
along the fluid flow direction of the infusion line by a fluid passage length comprised between 1 mm and 30 mm, and
radially by a diameter comprised between 0.2 mm and 3 mm;
wherein the damper passage section is constant along said fluid passage length.

69. A disposable set for an extracorporeal blood treatment apparatus, the disposable set comprising:
a filtration unit;
a blood circuit comprising:
a blood withdrawal line extending between a first end connected to the filtration unit and a second end,
a blood return line extending between a first end connected to the filtration unit and a second end;
the blood withdrawal line including a blood pump tract configured to be engaged by a blood pump of the extracorporeal blood treatment apparatus which is configured to determine a blood flow, wherein, at least during an operating condition, a return negative pressure is experienced downstream the filtration unit, said blood flow in the blood circuit being in a direction from the blood withdrawal line towards the filtration unit and from the filtration unit through the blood return line;
a post infusion line extending between a first end connected to the blood return line at a fluid access downstream the blood pump tract, and a second end configured to connect to an infusion substance source;
wherein the disposable set further comprises a pressure damper arranged proximate or at the first end of the post infusion line, the pressure damper configured to prevent or reduce passage of the return negative pressure to the post infusion line upstream the pressure damper, wherein the pressure damper comprises a one-way valve configured to allow fluid passage only in an infusion direction directed from the post infusion line towards the return line, and is configured to move between an open position, in which the fluid passage is allowed in said infusion direction, and a closed position in which fluid passage is prevented in both directions,
the one-way valve being preset at an opening pressure threshold to switch between the closed position and the open position and vice versa, so that:
when a differential pressure between an upstream section and a downstream section of the one-way valve is equal or higher than said opening pressure threshold, the one-way valve is configured to switch to or to maintain the open position, and
when said differential pressure is lower than said opening pressure threshold, the one-way valve is configured to switch to or to maintain the closed position.

70. Disposable set according to claim 69, wherein the differential pressure is defined between a high pressure zone upstream the one-way valve, and a low pressure zone downstream the one-way valve according to the infusion direction, said high pressure being higher than said low pressure, and wherein the one-way valve prevents a fluid passage in a direction from the return line to the post infusion line any conditions, wherein the preset opening pressure threshold of the one-way valve corresponds to a differential pressure value higher than 160 mmHg.

71. Disposable set according to claim 69, wherein said one-way valve is arranged on the post infusion line at a distance from the first end of the post infusion line no longer than 6 cm.

72. Disposable set according to claim 69, wherein the second end of the blood withdrawal line and the second end of the blood return line are configured to connect or connected to a blood duct of a further extracorporeal blood circuit of an Extra Corporeal Membrane Oxygenation (ECMO) circuit.

73. Disposable set according to claim 72, wherein the ECMO circuit comprises:
a blood withdrawal duct for connection with the vascular access of the patient and configured to receive blood from the patient,
a blood return duct for connection with the vascular access of the patient and configured to return blood to the patient,
a blood pump configured to promote blood flow in the ECMO circuit in a direction from the blood withdrawal duct towards the blood return duct, and through the oxygenator,
an oxygenator arranged on the blood return duct and configured to oxygenate the blood.

74. Disposable set according to claim 73, wherein the blood return line is connected to the ECMO circuit upstream the blood pump of the ECMO circuit and the pressure damper is located at the fluid access between the blood return line and the post infusion line, the pressure damper being arranged on the return line of the disposable set connected to the ECMO circuit where a negative pressure is experienced.
